SEQUENCE LISTING (19 sequences)

SEQ ID NO: 1 - DNA, 1059 bp, Homo sapiens
  source 1..1059: mol_type = other DNA
  CDS 1..1059
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 2 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - DNA, 951 bp, Homo sapiens
  source 1..951: mol_type = other DNA
  CDS 1..951
atgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggaactagaaccatga

SEQ ID NO: 4 - protein, 316 aa, Homo sapiens
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 5 - DNA, 765 bp, Homo sapiens
  source 1..765: mol_type = other DNA
  CDS 1..765
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 6 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 7 - DNA, 765 bp, synthetic construct
  source 1..765: mol_type = other DNA
atgtggcagctgctgctgccaaccgccctgctgctgctggtgtccgccggaatgaggacagaggacctgccaaaggccgtggtgtttctggagccccagtggtacagagtgctggagaaggactctgtgaccctgaagtgccagggcgcctattctcctgaggataacagcacacagtggtttcacaatgagagcctgatcagctcccaggcctctagctacttcatcgacgcagcaaccgtggacgattccggagagtatcggtgccagaccaacctgagcacactgtccgatccagtgcagctggaggtgcacatcggatggctgctgctgcaggcacctagatgggtgtttaaggaggaggaccccatccacctgcgctgtcacagctggaagaataccgccctgcacaaggtgacatacctgcagaacggcaagggcaggaagtacttccaccacaattctgacttttatatccccaaggccaccctgaaggattccggctcttatttctgccgcggcctggtgggcagcaagaacgtgtcctctgagaccgtgaatatcaccatcacacagggcctggccgtgtccacaatcagctccttctttccccctggctaccaggtgtctttctgtctggtcatggtgctgctgtttgccgtggacacaggcctgtatttctccgtgaagaccaacatccggtctagcacaagagactggaaggatcacaagttcaagtggcggaaggaccctcaggataagtga

SEQ ID NO: 8 - DNA, 7495 bp, synthetic construct
  source 1..7495: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 9 - DNA, 8085 bp, synthetic construct
  source 1..8085: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 10 - DNA, 7138 bp, synthetic construct
  source 1..7138: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 11 - DNA, 8639 bp, synthetic construct
  source 1..8639: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 12 - DNA, 7704 bp, synthetic construct
  source 1..7704: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 13 - DNA, 7250 bp, synthetic construct
  source 1..7250: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 14 - DNA, 7417 bp, synthetic construct
  source 1..7417: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 15 - DNA, 8084 bp, synthetic construct
  source 1..8084: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaacaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 16 - DNA, 7756 bp, synthetic construct
  source 1..7756: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcaagtttgtacaaaaaagcaggctgccaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 17 - DNA, 8767 bp, synthetic construct
  source 1..8767: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcaagtttgtacaaaaaagcaggctgccaccatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctggaactagaaccaggaagcggagagggcaggggaagtcttctaacatgcggggacgtggaggaaaatcccggccccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 18 - DNA, 8476 bp, synthetic construct
  source 1..8476: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcactgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 19 - DNA, 8203 bp, synthetic construct
  source 1..8203: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagccacgaacttctctctgttaaagcaagcaggagatgttgaagaaaaccccgggcctatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattctaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

(19) United States
(12) Patent Application Publication
(10) Pub. No.: US 2026/0201334 A1
(43) Pub. Date: Jul. 16, 2026

(54) METHODS OF PRODUCING MODIFIED NATURAL KILLER CELLS AND METHODS OF USE

(71) Applicant: The United States of America, as represented by the Secretary, Dept. of Health and Human Services, Bethesda, MD (US)

(72) Inventors: Richard W. Childs, Rockville, MD (US); David S.J. Allan, Rockville, MD (US)

(73) Assignee: The United States of America, as represented by the Secretary, Dept. of Health and Human Services, Bethesda, MD (US)

(21) Appl. No.: 19/416,316

(22) Filed: Dec. 11, 2025

Related U.S. Application Data
(62) Division of application No. 16/760,760, filed on Nov. 1, 2018, now Pat. No. 12,516,292.
Application No. 16/760,760 is a continuation-in-part of application No. 15/801,085, filed on Nov. 1, 2017, now Pat. No. 11,293,010.
Application No. 15/801,085 is a continuation-in-part of application No. PCT/US2017/043774, filed on Jul. 25, 2017.
(60) Provisional application No. 62/366,493, filed on Jul. 25, 2016.

(51) Int. Cl. C12N 5/0783; A61K 40/15; A61K 40/42; C07K 14/55; C07K 14/705; C12N 15/86

(52) U.S. Cl. CPC C12N 5/0646; A61K 40/15; A61K 40/42; A61K 2239/31; A61K 2239/38; A61K 2239/48; C07K 14/55; C07K 14/70596; C12N 15/86; C12N 2310/11; C12N 2310/122; C12N 2320/31; C12N 2501/20; C12N 2501/2302; C12N 2501/2315; C12N 2501/999; C12N 2510/00

(57) ABSTRACT

Disclosed herein are method of producing NK cells that include one or more heterologous nucleic acids. The methods include culturing a population of isolated NK cells in the presence of one or more cytokines to produce a population of activated NK cells. The population of activated NK cells are transduced with a viral vector comprising the one or more heterologous nucleic acids, for example by contacting the activated NK cells with viral particles including the viral vector. The resulting transduced NK cells are then cultured in the presence of one or more cytokines, and optionally in the presence of irradiated feeder cells, to produce a population of expanded transduced NK cells. Also disclosed are methods of treating a subject with a disorder (such as a tumor or hyperproliferative disorder) by administering to the subject NK cells produced by the methods described herein.

### CROSS REFERENCE TO RELATED APPLICATIONS

This application is a divisional of U.S. application Ser. No. 16/760,760, filed Apr. 30, 2020, which is the § 371 U.S. National stage of International Application No. PCT/US2018/058747, filed Nov. 1, 2018, which claims the benefit of U.S. application Ser. No. 15/801,085, filed Nov. 1, 2017, which is a continuation-in-part of International Application No. PCT/US2017/043774, filed Jul. 25, 2017, which claims the benefit of U.S. Provisional Application No. 62/366,493, filed Jul. 25, 2016, all of which are herein incorporated by reference in their entirety.

### FIELD

This disclosure relates to methods of producing modified natural killer cells, compositions comprising the modified natural killer cells, and methods of their use.

### SEQUENCE LISTING INCORPORATION BY REFERENCE

The Sequence Listing is submitted as an XML file in the form of the file named 4239-96570-24_Sequence_Listing.xml, which was created on Dec. 11, 2025, and is117,190 bytes, which is incorporated by reference herein.

### BACKGROUND

Besides antigen-specific cytotoxic T lymphocytes, cellular components of the innate immune system can contribute to immune surveillance of malignant cell growth. In particular, natural killer (NK) cells can eliminate abnormal cells without priming or sensitization. Their activity is determined by the balance of signals from inhibitory and activating NK cell receptors. Inhibitory receptors, such as killer immunoglobulin receptors (KIRs), interact with self major histocompatibility complex (MHC) class I antigens and protect normal cells from NK cell attack.

Efforts at harnessing the anti-tumor activity of NK cells have been investigated for the immunotherapy of human cancer for over two decades. However, many malignant cells express MHC class I antigens and are thus naturally resistant to lysis by autologous NK cells. Accordingly, the first clinical trials using adoptive transfer of autologous NK cells have failed to produce significant therapeutic effects. Modulation of NK cell cytokine, chemokine, and activation/inhibitory receptor expression is an attractive strategy to bolster NK cell anti-tumor activity. However, efficient genetic modification of primary NK cells has been difficult to achieve.

### SUMMARY

There is a need for simple and efficient gene transfer methods to effectively deliver and express genes of interest in primary NK cells. Disclosed herein are efficient viral vector-based methods for gene transfer into NK cells that demonstrate stable and robust long-term expression of transgenes.

Disclosed herein are method of producing NK cells that include or express one or more heterologous nucleic acids (referred to herein in some examples as “modified” NK cells). The methods include culturing a population of isolated NK cells (such as NK cells isolated or purified from a subject) in the presence of one or more cytokines (for example, interleukin (IL)-2, IL-15, and/or IL-21), and optionally in the presence of feeder cells, to produce a population of activated NK cells. In some examples, the population of isolated NK cells is cultured in the presence of IL-2 to produce the population of activated NK cells. In particular examples, the population of isolated NK cells is cultured in the presence of IL-2 and no other added cytokines to produce the population of activated NK cells. The population of activated NK cells is transduced with a viral vector including the one or more heterologous nucleic acids, for example by contacting the activated NK cells with viral particles including the viral vector. The resulting transduced NK cells are then cultured in the presence of one or more cytokines (for example, IL-2, and in some examples, IL-2 and no other added cytokines), and optionally in the presence of feeder cells, to produce a population of expanded transduced NK cells.

Also disclosed herein are methods of treating a subject with a disorder (such as a tumor or hyperproliferative disorder or a viral infection) with the modified NK cells. In some embodiments, the methods include obtaining a population of isolated NK cells from the subject or from a donor and culturing the population of isolated NK cells in the presence of one or more cytokines (for example, IL-2, IL-15, and/or IL-21), and optionally in the presence of feeder cells, to produce a population of activated NK cells. In some examples, the population of isolated NK cells is cultured in the presence of IL-2 to produce the population of activated NK cells. In particular examples, the population of isolated NK cells is cultured in the presence of IL-2 and no other added cytokines to produce the population of activated NK cells. The population of activated NK cells is transduced with a viral vector including one or more heterologous nucleic acids suitable for treating the subject's disorder, for example by contacting the activated NK cells with viral particles including the viral vector. The resulting transduced NK cells are then cultured in the presence of one or more cytokines (for example, IL-2, and in some examples, IL-2 and no other added cytokines), and optionally in the presence of feeder cells, to produce a population of expanded transduced NK cells, which are administered to the subject (for example, in a composition including a pharmaceutically acceptable carrier).

Also disclosed herein are modified NK cells (such as a population of modified NK cells) including one or more heterologous nucleic acids, such as NK cells transduced with a viral vector (such as a lentiviral vector) that includes one or more nucleic acids of interest. In some examples, the heterologous nucleic acid encodes CD34, CD4, CD19, CXCR4, CCR7, CXCR3, or CD16 (for example, high affinity CD16 or CD16-V158). Additional nucleic acids of interest are shown in Table 1, below. Also disclosed are compositions that include modified NK cells (such as a population of modified NK cells) and a pharmaceutically acceptable carrier. In some embodiments, the modified NK cells are produced by the methods disclosed herein.

The foregoing and other features of the disclosure will become more apparent from the following detailed description, which proceeds with reference to the accompanying figures.

### BRIEF DESCRIPTION OF THE DRAWINGS

FIGS. 1A and 1B are schematic diagrams illustrating an exemplary three plasmid lentiviral vector system that can be utilized in the disclosed methods (FIG. 1A) and an exemplary method for producing non-replicating lentivirus that can be used to transduce target cells (FIG. 1B).

FIG. 2 is a schematic diagram illustrating an exemplary method for producing a population of expanded transduced NK cells.

FIG. 3 is a series of panels showing expression of enhanced green fluorescent protein (eGFP) expression in CD56+ NK cells cultured in media and primed with IL-2 (500 IU/ml) prior to transduction (Condition 1) or CD56+ NK cells cultured on irradiated lymphoblastoid (LCL) cells and primed with interleukin-2 (IL-2; 500 IU/ml) prior to transduction (Condition 2). Analysis was by fluorescence-activated cell sorting (FACS) seven days post-transduction.

FIG. 4 is a graph showing transduction efficiency in CD56+ NK cells cultured in media containing IL-2 (500 IU/ml) for the indicated number of days prior to transduction. Two days after transduction, viral particles were removed and irradiated LCLs were added (LCL:NK ratio 10:1) with 500 IU/ml IL-2. eGFP expression was analyzed by FACS seven days post-transduction.

FIGS. 5A and 5B are graphs showing persistence of transgene expression (FIG. 5A) and cell viability (FIG. 5B) post-transduction in NK cells primed with 500 IU/ml IL-2 for three days prior to transduction.

FIG. 6 is a graph showing transduction efficiency in CD56+ NK cells cultured on irradiated LCLs in media containing 500 IU/ml IL-2 for the indicated number of days prior to transduction. Two days after transduction, viral particles were removed and the cells were maintained in media containing 500 IU/ml IL-2. eGFP expression was analyzed by FACs seven days post-transduction.

FIG. 7 is a series of graphs showing persistence of eGFP expression in transduced NK cells (top) and cell viability (bottom) at the indicated number of days post-transduction. Cells were cultured in medium containing 500 IU/ml IL-2 and irradiated feeder cells for 0, 3, or 7 days prior to transduction. eGFP expression was analyzed by FACS and cell viability was determined by trypan blue.

FIGS. 8A and 8B are a series of panels showing transduction efficiency of primary peripheral blood NK cells from three subjects (NK-1, NK-2, NK-3), expanded with irradiated LCL and 500 IU/ml IL-2 for 14 days prior to transduction. FIG. 8A shows FACS analysis of each cell line seven days post-transduction. FIG. 8B is a graph quantitating percent of CD56+ eGFP+ NK cells on the indicated number of days post-transduction.

FIG. 9 is a series of panels showing eGFP expression in NK cells primed with 500 IU/ml IL-2 (top row), 1000 IU/ml IL-2 (second row), 10 ng/ml IL-15 (third row), or 500 IU/ml IL-2 plus 10 ng/ml IL-15 (bottom row) for three days prior to transduction. Two days post-transduction, viral particles were removed and cells were maintained on irradiated LCLs (10:1 LCL:NK) with 500 IU/ml IL-2. eGFP expression was analyzed by FACS two weeks post-transduction.

FIG. 10 is a graph showing persistence of transgene expression at the indicated number of days post-transduction with the indicated treatment for three days prior to transduction.

FIG. 11 is a series of panels showing expression of the indicated receptors on NK cells primed with 500 IU/ml IL (top) or 500 IU/ml IL-2 plus 10 ng/ml IL-15 (bottom) for three days prior to transduction. Two days post-transduction, viral particles were removed and irradiated LCLs (10:1 LCL:NK cells) were added and cells were maintained in media with 500 IU/ml IL-2. Analysis was by FACS at two weeks post-transduction. Open traces, IsoAb, filled traces, Ag-specific Ab.

FIG. 12 is a series of panels showing expression of the indicated receptors on NK cells primed with 500 IU/ml IL (top) or 500 IU/ml IL-2 plus 10 ng/ml IL-15 (bottom) for three days prior to transduction. Two days post-transduction, viral particles were removed and irradiated LCLs (10:1 LCL:NK cells) were added and cells were maintained in media with 500 IU/ml IL-2. Analysis was by FACS at two weeks post-transduction.

FIG. 13 is a series of graphs showing tumor cell lysis of K562 cells (top) or MOLM14 cells (bottom) in NK cells primed with the indicated conditions for three days prior to transduction. Cells were expanded with irradiated LCLs for 14 days prior to testing their tumor killing capacity by ⁵¹Cr release assay.

FIG. 14 is a graph showing fraction of NK cells expressing GFP (circles) and cell viability (squares) versus the number of days of cytokine stimulation (500 IU/ml IL-2) prior to transduction. A balance of transgene expression and viability was achieved with 2-3 days of cytokine stimulation prior to transduction (boxed area).

FIG. 15 is a graph showing the fraction of NK cells expressing GFP in NK cells treated with the indicated cytokines prior to transduction. There was no significant difference between IL-2 alone and combinations of IL-2 with IL-15 and/or IL-21.

FIGS. 16A and 16B are graphs showing the effect of transduction reagents on transduction efficiency (FIG. 16A) and NK cell expansion (FIG. 16B).

FIG. 17 is a graph showing transduction efficiency of NK cells transduced with GFP under the control of the indicated promoters at MOI of 5, 20, or 100.

FIG. 18 is a graph showing the fold-change in expression of GFP in NK cells transduced with GFP under the control of the indicated promoters on days 7 and 14 post-transduction, compared to expression on day 0 (100%).

FIG. 19 is a graph showing activity (% degranulation) of NK cells transduced with GFP under the control of the indicated promoters or mock transduced cells after 14 days of expansion when contacted with K562 cells. P/I indicates treatment with PMA/ionomycin to show maximal degranulation capacity.

FIG. 20 is a graph showing activity of NK cells transduced with GFP under the control of the indicated promoters compared to untransduced cells after 14 days of expansion when contacted with K562 cells. P/I indicates treatment with PMA/ionomycin to show maximal degranulation capacity.

FIGS. 21A and 21B are schematic diagrams of a lentiviral vector for expression of CXCR4 (FIG. 21A) or a lentiviral vector for expression of CD34t and CXCR4 (FIG. 21B).

FIGS. 22A and 22B are plots showing CXCR4 and CD34 expression in NK cells transduced with a lentiviral vector encoding CXCR4 (FIG. 22A) or a lentiviral vector encoding both CD34t and CXCR4 (FIG. 22B).

FIG. 23 is a graph showing fold-expansion of NK cells activated in the presence or absence of LCL feeder cells, followed by viral transduction and expansion in the presence or absence of LCL feeder cells.

FIGS. 24A and 24B show results from NK cells from PBMC cultured with 500 U/ml IL-2 for 0 to 5 days as indicated, before transduction with pLV [Exp]-PGK>EGFP. Three days after transduction, cells were assayed for GFP expression and viability by flow cytometry. FIG. 24A shows typical gating of GFP+ NK cells. FIG. 24B shows mean and SEM (of logo transformed values for geometric mean fluorescence intensity (GMFI) GFP) of experiments from 3 donors. * indicates p<0.05 compared to cells without IL-2 stimulation (day 0) by Dunnett's multiple comparisons test after one-way ANOVA. (% Annexin⁻PI⁻ cells depicted in FIG. 24B were tabulated on gated CD56⁺CD3⁻ cells).

FIGS. 25A-25D are graphs showing GFP expression in human NK cells stimulated with IL-2 for three days before transduction with lentiviral vectors with the indicated promoters and GFP. Irradiated SMI-LCL feeder cells were added to the transduced NK cells and co-cultured for an additional 14 days (20 days total). Three days after transduction, NK cells were examined by flow cytometry (FIG. 25A) and transduction efficiency (FIG. 25B) and level of transgene expression were calculated (FIG. 25C). Stability of transgene expression in NK cells over time was quantified by dividing the day 6 EGFP % by the day 20 EGFP % and expressed as a percent (FIG. 25D). Mean and SEM (of logo transformed values for GMFI) are shown in each case for experiments from 4 donors. * indicates p<0.05 compared to the previously used PGK promotor containing vector by Dunnett's multiple comparisons test after one-way ANOVA.

FIGS. 26A and 26B show the effect of arrangement of insert sequences on transduction efficiency. NK cells from PBMC were stimulated with IL-2 for 3 days before transduction with lentiviral vectors containing the indicated insert sequences encoding combinations of PGK promotor, GFP, P2A-linker, IRES, truncated CD34 protein, and codon-optimized truncated CD34 protein (CD34opt). After 3 additional days, NK cells were measured by flow cytometry for GFP and surface expression of CD34 (FIG. 26A). Mean and SEM of experiments from 3 donors is shown. * indicates p<0.05 for selected comparisons by Tukey's multiple comparisons test after one-way ANOVA. Typical staining comparing constructs containing original versus codon-optimized truncated CD34 (FIG. 26B).

FIGS. 27A-27D show the effects of pre-stimulation of NK cells in the presence of feeder cells prior to lentiviral transduction. NK cells from PBMC were stimulated with IL-2 or IL-2+SMI-LCL feeder cells for 3 days. NK cells were removed from co-culture with anti-CD56 beads before transduction with pLV-EFS-GFP-2A-CD34opt viral particles at 20:1 MOI. After 3 additional days, SMI-LCL were added to both conditions and NK cells cultured until day 21 from the start of experiment. NK cells were examined by flow cytometry for GFP and CD34 transgene expression (FIG. 27A). Mean and SEM are shown for experiments from 4 donors. NK cell numbers were counted frequently throughout culture, and the fold expansion calculated (FIG. 27B). Mean and SEM (of log 10 transformed values) are shown in comparison with control expansions of un-transduced NK cells from the same donors, which received SMI-LCL on day 0 only. Overall yield of transduced cells relative to input was calculated by multiplying the fold expansion of all cells X the GFP⁺CD34⁺ fraction for each condition (FIG. 27C). Mean and SEM (of log 10 transformed values) are shown. Similar experiments were performed to determine the optimal period (2-7 days) of stimulation with SMI-LCL before transduction (pLV-EFS-GFP-2A-CD34opt at 20:1 MOI) (FIG. 27D). The percentage of transduced cells, fold expansion of cells, and overall yield of transduced cells relative to input were determined for experiments from 3 donors.

FIGS. 28A and 28B show the effect of including BX795 during transduction of NK cells pre-stimulated with LCL cells. NK cells from PBMC were stimulated with SMI-LCL feeder cells plus IL-2 for 5 days. NK cells were removed from co-culture with anti-CD56 beads before transduction with pLV-EFS-GFP-2A-CD34opt viral particles in the presence of varying doses of BX795 as indicated. After one day, NK cells were exchanged into medium without BX795. After 2 additional days, SMI-LCL were added again and NK cells cultured until day 21 from the start of experiment, monitoring cell numbers throughout. NK cells were assayed for GFP and CD34 expression by flow cytometry (FIG. 28A). The overall yield of transduced cells relative to input cells was calculated by multiplying fold expansion of cells X fraction of GFP⁺CD34⁺ cells. Mean and SEM are shown (of log₁₀ transformed values for fold expansions) for experiments from three donors. * indicates p<0.05 compared to conditions lacking drug by Dunnett's multiple comparisons test after one-way ANOVA. Similar experiments were performed to compare cells stimulated for 5 days with IL-2 or IL-2+SMI-LCL feeder cells before transduction with or without addition of 1.5 μM BX795 (FIG. 28B). The overall yield of transduced cells relative to input cells was tabulated as above.

FIGS. 29A and 29B show lentiviral expression of CD34opt, CD19, and CD4 from an optimized SMI-LCL protocol. NK cells were isolated from PBMC via anti-CD3 MACS bead depletion followed by anti-CD56 MACS bead positive selection. Cells were stimulated with SMI-LCL plus IL-2 for 4-5 days. Resulting cells were transduced in the presence of 1.5 μM BX795 with lentiviral vectors encoding codon optimized truncated CD34, truncated CD19, or truncated CD4 as indicated. After 1 day, cells were exchanged into medium without BX795. After 2 additional days, transduced NK cells were selected using MACS beads recognizing CD34, CD19, or CD4. Positively selected NK cells were cultured with SMI-LCL and proliferation of cells followed until 21 days from the start of the experiments. Flow cytometry evaluation of GFP and transgene expression before and after MACS bead selection (FIG. 29A). Cell numbers were monitored throughout and the overall yield of transduced cells relative to input cells calculated by multiplying the fold expansion of cells X the fraction positive for both GFP and transgene expression (FIG. 29B). Mean and SEM (of log 10 transformed values) of experiments from 3 donors are shown.

FIG. 30 shows degranulation (top and bottom left) and IFN-γ (top and bottom middle) and TNF-α (top and bottom right) production of NK cells transduced and expanded as in FIGS. 29A and 29B. Percentages were normalized to values measured with control un-transduced NK cells from the same donors expanded with SMI-LCL.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1 and 2 are exemplary CXCR4 nucleic acid and amino acid sequences, respectively.

SEQ ID NOs: 3 and 4 are exemplary truncated CD34 nucleic acid and amino acid sequences, respectively.

SEQ ID NOs: 5 and 6 are exemplary high affinity CD16 nucleic acid and amino acid sequences, respectively.

SEQ ID NO: 7 is an exemplary codon-optimized high affinity CD16 nucleic acid.

SEQ ID NO: 8 is an exemplary lentiviral expression vector including human cytomegalovirus (CMV) immediate early promoter and EGFP.

SEQ ID NO: 9 is an exemplary lentiviral expression vector including human eukaryotic translation elongation factor 1α (EF1A) promoter and EGFP.

SEQ ID NO: 10 is an exemplary lentiviral expression vector including a short version of human EF1A promoter (EFS) and EGFP.

SEQ ID NO: 11 is an exemplary lentiviral expression vector including human CMV early enhancer fused with chicken β-actin (CAG) promoter and EGFP.

SEQ ID NO: 12 is an exemplary lentiviral expression vector including a short version of CAG promoter and EGFP.

SEQ ID NO: 13 is an exemplary lentiviral expression vector including simian SV40 early promoter and EGFP.

SEQ ID NO: 14 is an exemplary lentiviral expression vector including mouse phosphoglycerate kinase 1 (PGK) promoter and EGFP.

SEQ ID NO: 15 is an exemplary lentiviral expression vector including human ubiquitin C promoter and EGFP.

SEQ ID NO: 16 is an exemplary lentiviral expression vector including mouse PGK promoter and human CXCR4.

SEQ ID NO: 17 is an exemplary lentiviral expression vector including mouse PGK promoter and truncated human CD34, T2A peptide, and human CXCR4.

SEQ ID NO: 18 is an exemplary lentiviral expression vector including short version of human EF1A promoter, EGFP, and truncated human CD4.

SEQ ID NO: 19 is an exemplary lentiviral expression vector including short version of human EF1A promoter, EGFP, and truncated human CD19.

### DETAILED DESCRIPTION

Disclosed herein are methods for efficiently and stably expressing a transgene in NK cells. The methods include an efficient viral vector-based method for gene transfer into NK cells and demonstrate stable and long-term robust expression of transgenes. High gene transfer rates into primary cells being transduced and the ability to produce high titers of virus particles for large-scale transduction of patient cells are important criteria for clinical trials. Lentiviral vectors, such as those utilized in examples herein, can be produced in high titer and concentrated without compromising their transduction efficiency. Additionally, the currently described method is cheaper and simpler to apply clinically as it does not require the need for several cumbersome and expensive steps adopted in current retroviral vector-mediated gene transfer protocols to obtain moderate transduction efficiency such as use of retronectin, spinoculation, and repeated/multiple transductions that might have a significant deleterious impact on the viability of the primary NK cells being transduced. In addition protocols for efficient ex vivo expansion of NK cells under Good Manufacturing Practice (GMP) conditions for adoptive NK cell based immunotherapy applications are available. The present highly efficient lentiviral vector-based gene transfer protocol can be used to complement a number of current ex vivo NK cell expansion protocols to generate large numbers of genetically reprogrammed NK cells, potentially revolutionizing NK cell based immunotherapeutic approaches by enhancing their antitumor efficacy in patients with cancer or viral infection.

The disclosed methods have several advantages over other methodologies for lentiviral vector-mediated genetic manipulation of human primary NK cells. The present lentiviral vector-based approach results in an efficient, robust, and highly reproducible method of stable gene transfer into primary human peripheral blood-derived NK cells. This is in contrast to episomal vectors that are lost with cell division/long-term culture, poxvirus vectors that inhibit nuclear function and eventually instigate host cell lysis, and the other non-viral-mediated gene delivery methods such as electroporation that allow only transient expression of the introduced genes. Furthermore, unlike gammaretroviral vectors, which integrate preferentially near transcription start sites and potentially activate oncogenes by promoter activation from the long terminal repeats (LTRs), lentiviral vectors integrate preferentially within highly expressed genes. Moreover, lentiviral vectors used in the disclosed methods have improved safety features such as a split genome lentiviral packaging design and have a self-inactivating design of the transfer vector through deletions in the 3′ LTR, thereby reducing the potential for promoter activation.

Transduced primary NK cells obtained by the methods disclosed herein are phenotypically and functionally normal and therefore allow for a multitude of gene therapy and immunotherapy applications. Importantly, this current transduction method is simple and involves in vitro culture of NK cells with one or more cytokines, followed by exposure to concentrated viral particles. Following transduction, cells are expanded in large numbers by co-culturing them with irradiated feeder cells in media containing one or more cytokines.

### I

Abbreviations

| | |
|---|---|
| CAR | chimeric antigen receptor |
| EBV-LCL | Epstein-Barr virus transformed |
| | lymphoblastoid cell line |
| eGFP | enhanced green fluorescent protein |
| FACS | fluorescence-activated cell sorting |
| GMP | good manufacturing practices |
| IL | interleukin |
| LTR | long terminal repeat |
| MOI | multiplicity of infection |
| NK | natural killer cell |
| PBMC | peripheral blood monocyte cells |
| shRNA | short hairpin RNA |

### II. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Cancer: Also referred to herein as a “malignant tumor” or “malignant neoplasm.” Any of a number of diseases that are characterized by uncontrolled, abnormal proliferation of cells, the potential of cancer cells to spread locally or through the bloodstream and lymphatic system to other parts of the body (e.g., metastasize), as well as any of a number of characteristic structural and/or molecular features. A “cancer cell” is a cell having specific structural properties, lacking differentiation, and being capable of invasion and metastasis.

CD34: A cell surface glycoprotein that functions as a cell-cell adhesion molecule. CD34 is a single-pass transmembrane protein with a highly glycosylated extracellular domain, a transmembrane domain, and an intracellular signaling domain. CD34 is expressed on hematopoietic cells and plays a role in cell migration. Exemplary human CD34 sequences include GenBank Accession Nos. NM_001025109 and NM_001773 (nucleic acid sequences) and NP_001020280 and NP_001764 (amino acid sequences), all of which are incorporated herein by reference as present in GenBank on Jul. 25, 2017.

Contacting: Placement in direct physical association, including both a solid and liquid form. In one example, contacting includes association between a substance (such as a cytokine) in a liquid medium and one or more cells (such as NK cells in culture). Contacting can occur in vitro with isolated cells or tissue or in vivo by administering to a subject.

Culturing or Cell culture: Growth of a population of cells in a defined set of conditions (such as culture medium, extracellular matrix, temperature, and/or time of culture) in vitro. In some examples, a cell culture includes a substantially pure culture (for example, isolated NK cells). In additional examples a cell culture includes a mixed culture, such as co-culture of two or more types of cells (for example a culture of NK cells with feeder cells). In further examples, a cell culture includes cells grown in contact with an extracellular matrix.

Culture Medium: A synthetic set of culture conditions with the nutrients necessary to support the viability, function, and/or growth of a specific population of cells, such as NK cells. Culture media generally include components such as a carbon source, a nitrogen source and a buffer to maintain pH. Additional components in culture media also may include one or more of serum (such as heat-inactivated serum), cytokines, hormones, growth factors, protease inhibitors, protein hydrolysates, shear force protectors, proteins, vitamins, glutamine, trace elements, inorganic salts, minerals, lipids, and/or attachment factors.

Cytokine: Proteins made by cells that affect the behavior of other cells, such as lymphocytes. In one embodiment, a cytokine is a chemokine, a molecule that affects cellular trafficking. The term “cytokine” is used as a generic name for a diverse group of soluble proteins and peptides that act as humoral regulators at nanomolar to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor α (TNF-α), interleukin-2 (IL-2), interleukin-7 (IL-7), interleukin-15 (IL-15), interleukin-21 (IL-21), and interferon-γ (IFN-γ).

Effective amount: A quantity of a specified agent sufficient to achieve a desired effect, for example, in a subject being treated with that agent. In some examples, an effective amount of the modified NK cells disclosed herein is an amount sufficient to treat or inhibit a disease or disorder in a subject (such as a tumor, hyperproliferative disorder, or viral infection). In other examples, an effective amount is an amount of modified NK cells sufficient to reduce or ameliorate one or more symptoms of a disease or disorder in a subject. The effective amount (for example an amount ameliorating, inhibiting, and/or treating a disorder in a subject) will be dependent on, for example, the particular disorder being treated, the subject being treated, the manner of administration of the composition, and other factors.

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

Feeder cells: Cells that provide support for another cell type in ex vivo or in vitro culture. Feeder cells may provide one or more factors required for survival, growth, and/or differentiation (or inhibiting differentiation) of the cells cultured with the feeder cells. Typically feeder cells are irradiated or otherwise treated to prevent their proliferation in culture. In some examples disclosed herein, NK cells are cultured with feeder cells, such as irradiated EBV-transformed lymphoblast cells (e.g., EBV-LCL cells).

Hyperproliferative disease: A disease or disorder in which the cells proliferate more rapidly than normal tissue growth. Thus, a hyperproliferating cell is a cell that is proliferating more rapidly than normal cells. In some examples, a hyperproliferative disorder includes a malignant tumor or cancer, but may also include a benign tumor.

Inhibiting or treating a condition: “Inhibiting” a condition refers to inhibiting the full development of a condition or disease, for example, a tumor. Inhibition of a condition can span the spectrum from partial inhibition to substantially complete inhibition (e.g., including, but not limited to prevention) of the disease. In some examples, the term “inhibiting” refers to reducing or delaying the onset or progression of a condition. “Treatment” refers to a therapeutic intervention that ameliorates a sign or symptom of a disease or condition after it has begun to develop. A subject to be administered an effective amount of the disclosed NK cells can be identified by standard diagnosing techniques for such a disorder, for example, presence of the disease or disorder or risk factors to develop the disease or disorder.

Isolated: An “isolated” or “purified” biological component (such as a cell, nucleic acid, peptide, protein, protein complex, or virus-like particle) has been substantially separated, produced apart from, or purified away from other components (for example, other biological components in the cell or the organism in which the component naturally occurs). Cells, nucleic acids, peptides and proteins that have been “isolated” or “purified” thus include cells, nucleic acids, and proteins purified by standard purification methods.

The term “isolated” or “purified” does not require absolute purity; rather, it is intended as a relative term. Thus, for example, an isolated biological component is one in which the biological component is more enriched than the biological component is in its natural environment within a cell, organism, sample, or production vessel (for example, a cell culture system). Preferably, a preparation is purified such that the biological component represents at least 50%, such as at least 70%, at least 80%, at least 90%, at least 95%, or greater, of the total biological component content of the preparation.

Natural Killer (NK) cells: Cells of the immune system that kill target cells in the absence of a specific antigenic stimulus and without restriction according to MHC class. Target cells can be tumor cells or cells harboring viruses. NK cells are characterized by the presence of CD56 and the absence of CD3 surface markers. NK cells typically comprise approximately 10 to 15% of the mononuclear cell fraction in normal peripheral blood. Historically, NK cells were first identified by their ability to lyse certain tumor cells without prior immunization or activation. NK cells are thought to provide a “back up” protective mechanism against viruses and tumors that might escape the CTL response by down-regulating MHC class I presentation. In addition to being involved in direct cytotoxic killing, NK cells also serve a role in cytokine production, which can be important to control cancer and infection.

In some examples, a “modified NK cell” is a NK cell transduced with a heterologous nucleic acid (such as one or more of the nucleic acids or vectors disclosed herein) or expressing one or more heterologous proteins. The terms “modified NK cell” and “transduced NK cell” are used interchangeably in some examples herein.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compositions, such as one or more modified NK cells and/or additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Subject: A living multi-cellular vertebrate organism, a category that includes both human and non-human mammals (such as mice, rats, rabbits, sheep, horses, cows, and non-human primates).

Transduce: Transferring nucleic acid into a cell, such as transfer of a heterologous nucleic acid into a host cell. As used herein, the term transduce (or transfect or transform) includes all techniques by which a nucleic acid is introduced into a cell, including but not limited to transformation with plasmid vectors, infection with viral vectors or viral particles, and introduction of naked DNA by electroporation, nucleofection, lipofection, or particle gun acceleration.

Transgene: A heterologous nucleic acid introduced into a cell, for example, by transduction. In some examples, a transgene is a nucleic acid encoding a protein of interest. In other examples, a transgene is a nucleic acid that is capable of modulating expression of a nucleic acid of interest, such as a short hairpin RNA (shRNA), small interfering RNA (siRNA), or antisense nucleic acid. The transgene may be operably linked to one or more expression control sequences, for example, a promoter.

A “heterologous” nucleic acid or protein refers to a nucleic acid or protein originating from a different genetic source. For example, a nucleic acid or protein that is heterologous to a cell originates from an organism or individual other than the cell in which it is expressed. In other examples, a heterologous nucleic acid or protein originates from a cell type other than the cell in which it is expressed (for example, a nucleic acid or protein not normally present in NK cells is heterologous to NK cells). In further examples, a heterologous nucleic acid includes a recombinant nucleic acid, such as a protein-encoding nucleic acid operably linked to a promoter from another gene and/or two or more operably linked nucleic acids from different sources.

Vector: A nucleic acid molecule allowing insertion of foreign or heterologous nucleic acid into a cell without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and/or translation of an inserted gene or genes. In some non-limiting examples, the vector is a viral vector, such as a retroviral vector or lentiviral vector.

### II. Methods of Producing Modified NK Cells

Disclosed herein are methods of producing modified NK cells (such as NK cells including or expressing one or more heterologous nucleic acids or including all or a portion of a viral vector including one or more heterologous nucleic acids). In particular examples, the methods disclosed herein are utilized to transduce resting or short-term activated NK cells (rather than NK cell lines or expanded NK cells), which have previously been challenging to transduce with viral vectors at high efficiency.

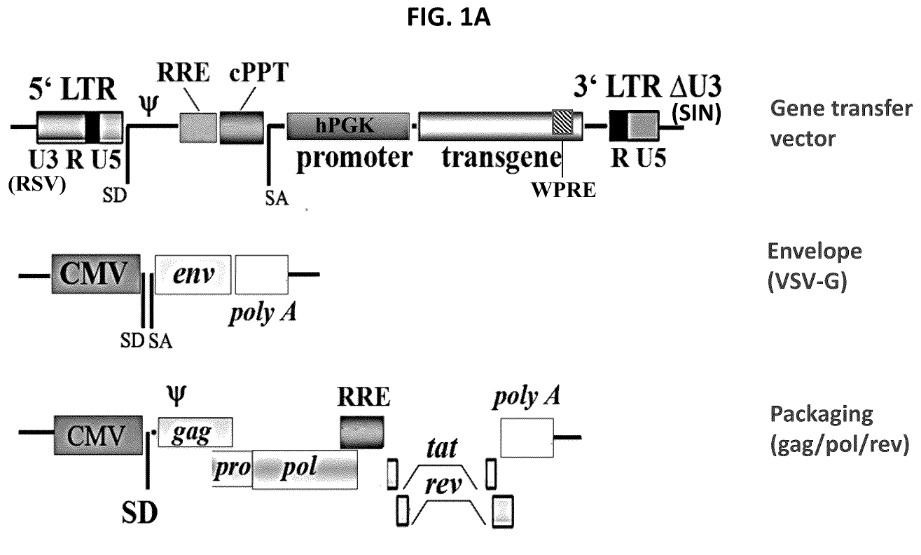

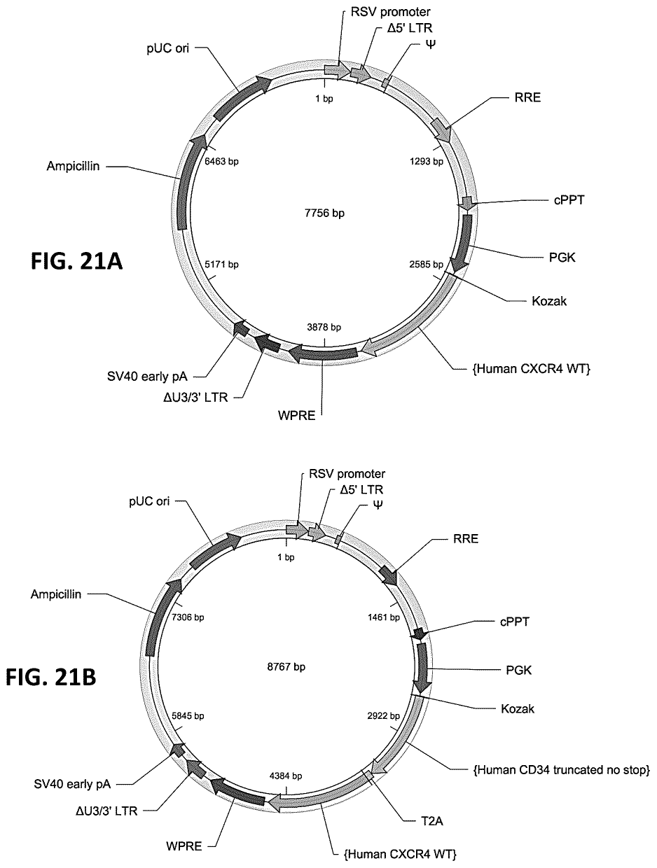

In some embodiments, the disclosed methods utilize a lentiviral system to introduce one or more heterologous nucleic acids (“transgenes”) into NK cells. Lentivirus systems for virus production for transduction are generally split across multiple plasmids to increase their safety. Exemplary lentiviral systems include “second generation” systems with three plasmids or “third generation” systems with four plasmids. An exemplary three plasmid system is illustrated in FIG. 1A. The transgene nucleic acid is included in a transfer plasmid and is operably linked to a promoter (in a non-limiting example, an hPGK promoter). The transfer plasmid also includes 5′ and 3′ LTRs and may include additional elements, such as the psi packaging system. Lentivirus proteins for producing viral particles (such as env, gag, pol, rev, and/or tat) are encoded by two or three separate plasmids. Exemplary transfer vectors are also shown in FIGS. 21A and 21B. The lentivirus plasmids (three or four plasmids) are transfected in a mammalian cell line, which produces lentivirus particles that are not capable of replication. The lentivirus particles are then used to transduce other cells (such as NK cells). An exemplary method for producing lentiviruses (such as non-replicating lentivirus) for use in the methods disclosed herein is shown in FIG. 1B.

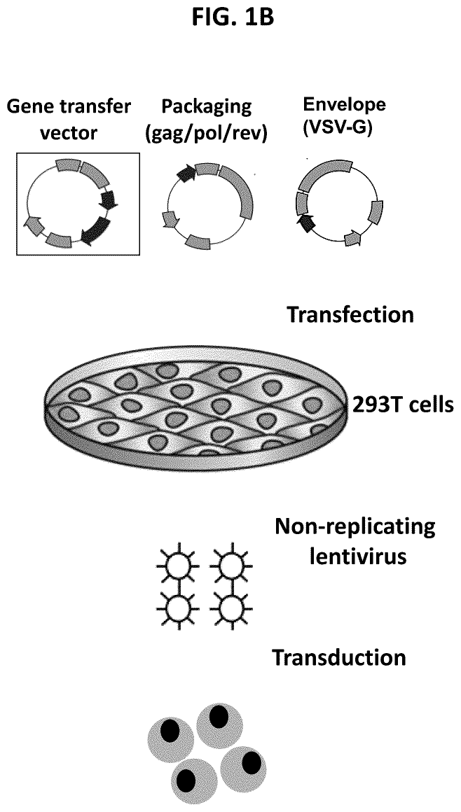

In some embodiments, disclosed herein are methods for producing modified NK cells utilizing transduction with a viral vector. An overview of an exemplary process is shown in FIG. 2. In some examples, the methods include obtaining or preparing purified NK cells and activating (or “priming”) the NK cells by culturing the purified NK cells in medium that includes one or more cytokines (such as IL-2, IL-15, and/or IL-21) for 1-14 days. The activated NK cells are transduced with a viral vector (such as a lentiviral vector) including one or more heterologous nucleic acids, for example, by incubating activated NK cells with the viral vector (for example, viral particles including the viral vector) for 1-3 days. The transduced NK cells are then expanded for 1-50 days (or more), for example, by culture with one or more cytokines (such as IL-2) and/or in the presence of feeder cells, to produce expanded modified NK cells. In some examples, the activated NK cells are transduced with a viral vector that includes a nucleic acid encoding a truncated CD34 protein (CD34t) lacking the intracellular signaling domain (for example a CD34t nucleic acid operably linked to another nucleic acid of interest). The CD34t protein includes the extracellular and transmembrane regions of CD34, and as a result, it is expressed on the cell surface, but does not affect activity of cells expressing the truncated protein (Norell et al., Cancer Immunol. Immunother. 59:851-862, 2010). To enrich for transduced cells, cells expressing CD34t (e.g., transduced cells) can be identified with an anti-CD34 antibody, and can be isolated using flow cytometry or immuno-magnetic methods. In other examples, the activated NK cells are transduced with a viral vector that includes a nucleic acid encoding a CD4 or CD19 protein (or a truncated CD4 or CD19 protein, for example, lacking the intracellular domain). To enrich for transduced cells, cells expressing CD4 or CD19 can be identified with an anti-CD4 or anti-CD19 antibody, and can be isolated using flow cytometry or immuno-magnetic methods. Methods for producing modified NK cells are discussed in more detail below.

In particular embodiments of the disclosed methods, purified or isolated NK cells are transduced prior to expansion and/or in the absence of feeder cells. By transducing NK cells prior to expansion, it is possible to reduce the amount of viral particles needed (such as reducing the amount of viral particles by least about 10-fold, 50-fold, 100-fold, 200-fold, 300-fold, 400-fold, 500-fold, 600-fold, 700-fold, 800-fold, 900-fold, 1000-fold, 2000-fold, 5000-fold, or 10,000-fold) than when transducing expanded NK cells. In addition, in some examples, it is simpler and/or requires less labor, cost, and/or time to transduce NK cells prior to expansion. In some examples, the inventors observed that when NK cells were contacted with viral particles in the presence of a feeder cell layer, the virus preferentially transduced the feeder cells, rather than the NK cells. Furthermore, in some examples, the transgene(s) may be toxic to the feeder cells, and may negatively impact NK cell expansion. Thus, in some non-limiting examples, NK cells are more efficiently transduced when feeder cells are not present.

In other embodiments, the NK cells are activated, transduced, and/or expanded in the presence of feeder cells (such as irradiated feeder cells). In some examples, NK cells are activated as described herein in the presence of feeder cells (e.g., at least 1:1 ratio (feeder cells:NK cells), for example, at least 2:1, 5:1, 10:1, 15:1, 20:1, or more) prior to transduction (for example for 1-5 days, such as 1, 2, 3, 4, or 5 days). The NK cells are then transduced and cultured for 1-5 days (such as 1, 2, 3, 4, or 5 days, for example for 3 days), still in the presence of feeder cells. The NK cells are then replated with feeder cells (for example, at least 2:1, 5:1, 10:1, 15:1, 20:1 ratio of feeder cells:NK cells) for expansion. In one non-limiting example, NK cells are isolated and plated with feeder cells at 10:1 feeder cells: NK cells. The NK cells are activated for 2-3 days with IL-2 prior to transduction. Three days after transduction, the NK cells are replated with 10:1 feeder cells: NK cells and continued to be cultured in the presence of IL-2 for expansion.

In other embodiments, the NK cells are activated in the presence of feeder cells. The feeder cells are substantially removed prior to transduction of the activated NK cells (for example, using immunomagnetic selection for CD19 or CD56). Following transduction, the NK cells are expanded in the presence of feeder cells. In some examples, NK cells are activated as described herein in the presence of feeder cells (e.g., at least 1:1 ratio (feeder cells:NK cells), for example, at least 2:1, 5:1, 10:1, 15:1, 20:1, or more) prior to transduction (for example for 1-5 days, such as 1, 2, 3, 4, or 5 days). The feeder cells are then separated from the activated NK cells prior to transduction. In some examples, the feeder cells are removed using immunomagnetic depletion for a cell surface protein specific to the feeder cells (e.g., CD19 for LCL feeder cells). Alternatively, the feeder cells can be separated from the NK cells using an NK cell surface specific protein, such as CD56. The separated NK cells are then transduced and cultured for 1-5 days (such as 1, 2, 3, 4, or 5 days, for example for 3 days), in the absence of feeder cells. The transduced NK cells are replated with feeder cells (for example, at least 2:1, 5:1, 10:1, 15:1, 20:1 ratio of feeder cells:NK cells) for expansion. In one non-limiting example, NK cells are isolated and plated with feeder cells at 10:1 feeder cells: NK cells. The NK cells are activated for 2-3 days with IL-2 prior to transduction. The feeder cells are removed using CD19 depletion and/or CD56 selection prior to transduction. Three days after transduction, the NK cells are replated with 10:1 feeder cells: NK cells and continued to be cultured in the presence of IL-2 for expansion.

The disclosed methods provide high efficiency transgene expression in the modified NK cells. In some embodiments, transgene expression in NK cells obtained with the disclosed methods is greater than 25%, such as at least 30%, at least 35%, at least 40%, at least 45%, at least 50%, at least 55%, at least 60%, at least 65%, at least 70%, at least 75%, at least 80%, such as 30-60%, 45-75%, 50-80%, 40-60%, or 40-50%. Transgene expression is also long-lasting post-transduction, for example for 1-10 weeks or more (such as 2-4 weeks, 3-6 weeks, 4-8 weeks, 7-10 weeks or more). In some examples, the transgene is expressed for at least 3 days, at least 5 days, at least 7 days, at least 10 days, at least 14 days, at least 21 days, at least 28 days, at least 35 days, at least 42 days, at least 49 days, at least 56 days, or more post-transduction. In other examples, the transgene expression continues for the lifetime of the NK cell either in vitro or following administration to a subject.

### A. Isolation and Enrichment of NK Cells

Techniques for the in vitro isolation and enrichment of NK cells are described herein. An exemplary procedure is described in US Pat. App. Publ. No. 2014/0086890, incorporated herein by reference in its entirety. One of ordinary skill in the art can identify additional methods for expanding NK cells, for example as described in Childs et al., Hematol. The Education Program 2013:234-246, 2013, incorporated herein by reference in its entirety.

Mononuclear cells are collected from a subject (such as a donor subject or a subject with a tumor or hyperproliferative disease or viral infection) or from a donor HLA-matched to the subject to be treated. In some examples, mononuclear cells are collected by an apheresis procedure. The mononuclear cells are enriched for NK cells, for example by negative depletion using an immuno-magnetic bead strategy. In some examples, NK cells are enriched by depleting the mononuclear cell sample of T cells, B cells, monocytes, dendritic cells, platelets, macrophages, and erythrocytes utilizing a mixture of biotinylated monoclonal antibodies. The non-NK cells in the sample are removed with magnetic beads coupled to streptavidin, resulting in an enriched preparation of NK cells. An exemplary commercially available kit for this method is Dynabeads® Untouched™ Human NK Cells kit (ThermoFisher Scientific, Waltham, MA). In another example, NK cells are enriched by positive selection of CD56⁺ NK cells, for example utilizing magnetic beads conjugated to an anti-CD56 antibody (such as CD56 MicroBeads, Miltenyi Biotec, Inc., Auburn, CA). In other examples, a two-step method including negative depletion (such as T cell depletion) followed by positive selection of CD56+ NK cells is used for enriching NK cells. These methods can be carried out under or adapted for Current Good Manufacturing Practice (cGMP). One of ordinary skill in the art can identify other methods that can be used to prepare an enriched population of NK cells.

Bulk NK cells or NK cell subsets isolated by additional enriching procedures, such as through the use of immuno-magnetic beads or flow sorting, may be grown in cell culture medium. In one example, the medium is Cellgro SCGM serum-free media (CellGenix, Gaithersburg, MD) containing 10% human AB serum, 50 U/mL penicillin, 50 μg/mL streptomycin, and 500 IU/mL IL-2 or in X-VIVO™ 20 media containing 10% heat inactivated human AB serum or 10% autologous serum.

The isolated NK cells can be analyzed by flow cytometry for the expression of markers such as CD56, CD16, TRAIL, FasL, NKG2D, LFA-1, perforin, and granzymes A and B. Chromium release assays can be used to assess NK cell cytotoxicity against cell targets. One of ordinary skill in the art can identify other methods to assess the isolated NK cell population (for example, purity, viability, and/or activity).

In some embodiments, enriched NK cells (typically >99% CD3 negative and >85% CD56+) are optionally also expanded in vitro. In one non-limiting example, the enriched NK cells are cultured with an irradiated EBV-LCL feeder cell line (such as SMI-LCL) in medium including 500 IU/ml IL-2 for up to 21 days. Utilizing this technique, expansions of NK cells in the range of 200- to 1000-fold may be achieved (expanded NK cells are typically >99% CD3 negative and >90% CD56+). In some examples, the starting population of enriched NK cells is about 0.8-1.6×10⁸ total NK cells, which over a 2-4 week period expand up to 1000-fold or greater in vitro. Similar numbers of NK cells have been expanded in scaled up experiments using GMP conditions. In some examples, NK cells are expanded in G-Rex® containers (Wilson Wolf, New Brighton, MN). The G-Rex®100 container supports NK expansions to doses of 2.5×10⁸ NK cells/kg or higher. NK cells cultured in G-Rex®100 containers could be cultured at concentrations up to 4×10⁶ NK cells/ml.

### B. NK Cell Activation

The disclosed methods include activation (also referred to herein as “priming”) of NK cells prior to transduction. Isolated NK cells (e.g., >95% CD3⁻ and >85% CD56⁺ or >99% CD3⁻ and >90% CD56⁺) are prepared as described in Section IIA or are otherwise obtained (for example, from a previous preparation, such as a cryopreserved isolated NK cell preparation). Cytokine stimulation induces metabolic activation and permits active gene expression in NK cells similar to other lymphocyte subsets, which may assist with efficient lentiviral vector mediated gene transduction.

In some embodiments of the disclosed methods, isolated NK cells (such as NK cells isolated from a subject or a donor or NK cells isolated and expanded from a subject or donor) are activated by culturing the isolated NK cells with one or more cytokines for a period of time prior to transduction. The NK cells are cultured in a culture medium including one or more cytokines for 1-14 days (such as 1-10 days, 1-7 days, 1-5 days, 2-6 days, 3-8 days, 1-4 days, or 1-3 days) prior to transduction. In some examples, the NK cells are cultured with one or more cytokines for 12 hours, 18 hours, or 1, 2, 3, 4, 5, 6, 7, 8, 9, 10, 11, 12, 13, or 14 days prior to transduction. In some examples, the NK cells are primed by culture with one or more cytokines in the absence of feeder cells. Optionally, the NK cells are primed by culture with one or more cytokines and irradiated feeder cells (such as those discussed in Section IIC) for 1-14 days prior to transduction.

The NK cells are cultured with one or more cytokines for 1-14 days prior to transduction. The one or more cytokines include IL-2, IL-15, and/or IL-21. In some examples, the NK cells are cultured in culture medium including IL-2 for 1-14 days prior to transduction (such as 1-7 days, 2-6 days, 5-10 days, or 7-14 days). In particular examples, the population of isolated NK cells is cultured in the presence of IL-2 and no other added cytokines to produce the population of activated NK cells. IL-2 is included in the culture medium at about 10-2000 IU/ml (such as about 50-100 IU/ml, 100-500 IU/ml, 200-600 IU/ml, 500-1000 IU/ml, or 1000-2000 IU/ml, for example, about 10 IU/ml, 20 IU/ml, 50 IU/ml, 100 IU/ml, 200 IU/ml, 500 IU/ml, 1000 IU/ml, 1500 IU/ml, 2000 IU/ml, or more). In some non-limiting examples, the NK cells are activated in culture medium including 500 IU/ml IL-2 or 1000 IU/ml IL-2 for 1-6 days, 2-3 days, 3-5 days, or 2 days.

In other examples, the NK cells are cultured in culture medium including IL-15 for 1-14 days prior to transduction (such as 1-7 days, 2-6 days, 5-10 days, or 7-14 days). IL-15 is included in the culture medium at about 1-100 ng/ml (such as about 1-10 ng/ml, 5-20 ng/ml, 10-50 ng/ml, 25-75 ng/ml, or 50-100 ng/ml, for example, about 1 ng/ml, 5 ng/ml, 10 ng/ml, 20 ng/ml, 30 40 ng/ml, 50 ng/ml, 60 ng/ml, 70 ng/ml, 80 ng/ml, 90 ng/ml, or 100 ng/ml). In one non-limiting example, the NK cells are activated in culture medium including 10 ng/ml or 50 ng/ml IL-15 for 1-6 days, 2-3 days, or 3-5 days.

In still further examples, the NK cells are cultured in culture medium including IL-2 and IL-15 for 1-14 days prior to transduction (such as 1-7 days, 2-6 days, 5-10 days, or 7-14 days), for example culture medium including 1-1000 IU/ml IL-2 and 1-100 ng/ml IL-15. In one non-limiting example, the NK cells are activated in culture medium including 500 IU/ml IL-2 and 10 ng/ml or 50 ng/ml IL-15 for 1-6 days, 2-3 days, or 3-5 days. In other examples, the NK cells are cultured in culture medium including IL-2 and IL-21 for 1-14 days prior to transduction (such as 1-7 days, 2-6 days, 5-10 days, or 7-14 days), for example culture medium including 1-1000 IU/ml IL-2 and 1-100 ng/ml IL-21. In one non-limiting example, the NK cells are activated in culture medium including 500 IU/ml IL-2 and 20 ng/ml IL-21 for 1-6 days, 2-3 days, or 3-5 days. In another example, the NK cells are cultured in culture medium including 1-1000 IU/ml IL-2, 1-100 ng/ml IL-15, and 1-100 ng/ml IL-21 for 1-14 days prior to transduction (such as 1-7 days, 2-6 days, 5-10 days, or 7-14 days). In one non-limiting example, the NK cells are activated in culture medium including 500 IU/ml IL-2, 10 ng/ml IL-15, and 20 ng/ml IL-21 for 1-6 days, 2-3 days, or 3-5 days.

In some embodiments, prior to transduction, the NK cells are also cultured in the presence of one or more inhibitors of innate immune signaling or viral sensing, such as an inhibitor of toll-like receptor (TLR) and/or RIG-I-like receptor (RLR), an inhibitor of TBK1/IKKε, an inhibitor of AIM2-like receptors (ALR), an inhibitor of cyclic-GMP-AMP (cGAS), and/or an inhibitor of stimulator of interferon genes (STING). Exemplary inhibitors of TLR, RLR, ALR, cGAS, and/or STING include ST2825, chloroquine, hydroxychloroquine, CpG-52364, SM934, IRS-954, DV-1179, IMO-3100, IMO-8400, IMO-9200, IHN-ODN 2088, IHN-ODN-24888, RU.521, PF-06928215, Pepinh-MYD, Pepinh-TRIF, Z-VAD-FMK, 2-aminopurine, BAY11-7082, Celastrol, CLI-095, H-89, Norharmane, and IRAK1/4 inhibitor (see e.g., Gao et al., Front. Physiol. 8:508, 2017; Vincent et al., Nat. Commun. 8:750, 2017; Sutlu et al., Human Gene Ther. 23:1090-1100, 2012; Hall et al., PLOS One 12:e0184843, 2017). Exemplary inhibitors of TBK1/IKKε include BX795, MRT67307, AZ13102909, MPI-0485520, Amlexanox, Compound II, SR8185 and CYT387 (see, e.g., Hasan et al., Pharmacol. Res. 111:336-342, 2016). In one non-limiting example, the inhibitor is BX795. In some examples, the NK cells are cultured in medium including the inhibitor (e.g., BX795) for 1-14 days prior to transduction (such as 1-7 days, 2-6 days, 5-10 days, or 7-14 days) in the presence or absence of feeder cells. In one example, the NK cells are cultured in medium including the inhibitor for about 1-24 hours (such as 1-8 hours, 6-18 hours, or 12-24 hours) prior to transduction. In one example, the inhibitor is BX795, such as about 0.25 μM to 15 μM (for example, about 0.25 μM to about 3 μM, about 0.5 μM to about 5 μM, about 1.5 μM to about 12 μM, about 0.75 μM to about 3 μM, or about 0.5 μM, about 0.75 μM, about 1.5 μM, about 3 μM, about 6 μM, or about 12 μM). In one non-limiting example, the inhibitor is 1.5 μM BX795.

### C. Transduction and Expansion of Transduced NK Cells

The activated (primed) NK cells, such as those described in Section IIB are transduced with a viral vector including one or more heterologous nucleic acids, such as one or more nucleic acids encoding a protein of interest or another nucleic acid of interest (such as an shRNA). In particular non-limiting examples, the vector is a lentiviral vector.

Viral vectors suitable for gene delivery to NK cells include retrovirus, adenovirus, adeno-associated virus, vaccinia virus, fowlpox, and lentivirus vectors. In particular non-limiting examples disclosed herein, NK cells are transduced with lentiviral vectors including one or more heterologous nucleic acids of interest. Some advantages of using a lentiviral system include long-term expression of the transgene, the ability to transduce both dividing cells and non-dividing cells, the ability to deliver complex genetic elements, lack of expression of viral proteins after transduction, lack of insertional mutagenesis in human cells, high titer production, and ease of vector manipulation and production.

Disclosed herein are lentiviral vectors or constructs including one or more heterologous nucleic acids of interest. In particular examples, the nucleic acid(s) of interest (such as those described in Section III) is included in a lentiviral gene transfer vector (see, e.g., FIG. 1A). The nucleic acid(s) of interest in the transfer vector is operably linked to one or more expression control elements, such as a promoter. Exemplary promoters include constitutive promoters such as cytomegalovirus (CMV), SV40, phosphoglycerate kinase (PGK), ubiquitin C (UBC), elongation factor-1 (EFS), chicken β-actin short promoter (CBH), EF-1 alpha (EF1a) promoter, or EF1a short promoter, a hybrid promoter (such as a CMV enhancer fused to chicken β-actin promoter (CAG)), or an inducible or tissue-specific promoter. In other examples, for example when the nucleic acid of interest is a shRNA, the nucleic acid may be operably linked to an RNA polymerase III promoter, such as a U6 or H1 promoter.

Additional expression control elements that may be included in the transfer vector include sequences that control or regulate transcription and/or translation of a nucleic acid, such as enhancers, leader sequences, transcription terminators, start and/or stop codons, internal ribosome entry sites (IRES), splicing signals, and polyadenylation signals. In examples where the vector or construct includes two (or more) heterologous nucleic acids of interest, the nucleic acids are operably linked, for example, separated by an IRES or other multicistronic element such as a P2A and/or T2A element. The vector may also contain additional elements such as packaging signals (e.g., lentivirus ψ packaging signal), a central polypurine tract (cPPT), a woodchuck hepatitis virus post-transcriptional regulatory element (WPRE), or a Rev Response element (RRE). In some examples, the lentivirus vector is self-inactivating.

Lentivirus vectors including one or more nucleic acids of interest can be prepared by one of ordinary skill in the art utilizing conventional molecular biology techniques. For example, the nucleic acid of interest can be cloned into a lentivirus transfer vector. Lentivirus plasmid systems (such as 3 or 4 plasmid systems) are commercially available, for example from Clontech (Mountain View, CA), ThermoFisher Scientific (Waltham, MA), or Addgene (Cambridge, MA). In some examples, lentivirus vectors are modified to suit a particular use, such as to obtain sustained expression in hematopoietic cells. In some examples, the modifications include one or more of the modifications described in Example 1, below.

In some embodiments, the lentiviral vector includes or consists of a nucleic acid sequence with at least 90% sequence identity (such as at least 90%, 91%, 92%, 93%, 94%, 95%, 96%, 97%, 98%, 99%, or 100% identity) to any one of SEQ ID NOs: 8-19. In some non-limiting examples, the lentiviral vector includes or consists of the nucleic acid sequence of any one of SEQ ID NOs: 8-19. A vector map of SEQ ID NO: 16 is shown in FIG. 21A and a vector map of SEQ ID NO: 17 is shown in FIG. 21B.

In some embodiments, the disclosed lentiviral vectors include a promoter operably linked to a nucleic acid(s) of interest. The promoter and/or the nucleic acid(s) of interest in SEQ ID NOs: 8-19 can be replaced with any promoter and/or nucleic acid(s) of interest. For example, the vectors of SEQ ID NOs: 16 and 17 include the PGK promoter (e.g., at nucleotide positions 1959-2469); however, this promoter could be replaced with a different promoter, such as the EFS promoter (e.g., nucleotides positions 1959-2190 of SEQ ID NO: 10). Alternatively, the nucleic acid linked to the promoter can be replaced with an alternative nucleic acid(s) for expression. In one example, the vector of SEQ ID NO: 10 includes the EFS promoter (e.g., at nucleotide positions 1959-2190) linked to a nucleic acid encoding EGFP (nucleotide positions 2221-2940). The nucleic acid encoding EGFP in the vectors disclosed herein can be replaced with a nucleic acid encoding a different nucleic acid of interest, including but not limited to a nucleic acid encoding CXCR4, CD16, CD34t, (nucleic acid positions in SEQ ID NOs: 16 and 17 are shown Table 3), CD4 (or truncated CD4), CD19 (or truncated CD19) or a nucleic acid encoding other nucleic acid(s) of interest, such as the transgenes shown in Table 1. In particular examples, the nucleic acid of interest is operably linked to a PGK, EFS, or SV40 promoter. Other elements of the disclosed vectors (shown in Table 4) can also be changed (for example, replaced) or modified, as desired.

Lentivirus particles are produced by transfecting a mammalian cell line (such as 293T cells or a derivative thereof) with the plasmids of the lentivirus system (such as the three plasmid system illustrated in FIG. 1A). Transfection is carried out by standard methods, for example utilizing calcium-phosphate or polyethylenimine-mediated transfection or commercially available transfection reagents such as Lipofectamine® (ThermoFisher Scientific, Waltham, MA), FuGene® (Promega, Madison, WI), Universal Transfection Reagent (Sigma-Aldrich, St. Louis, MO), or SuperFect® (Qiagen, Valencia, CA) transfection reagents. Following transfection, lentiviral particles are released into the culture medium and are harvested. In some examples, the transfected cells are cultured for about 18-72 hours (for example, about 18-36 hours, 24-48 hours, or 36-72 hours, such as 18, 24, 36, 48, 60, or 72 hours). The virus-containing medium, which contains the packaged lentivirus particles, is harvested. In some examples, the virus is concentrated (such as by about 2-fold, 5-fold, 10-fold, 20-fold, 50-fold, 100-fold, or more) for example, by ultracentrifugation of the culture supernatant. The virus preparation can be analyzed to determine the number of viral particles in a particular volume (e.g., pfu/ml, for example by p24 ELISA) or to determine the number of particles including the RNA of interest (e.g., transducing units (TU)/ml, for example by FACS analysis). In some examples, the concentrated virus preparation contains about 10⁷-10¹⁰ TU/ml (e.g., about 10⁷-10⁹, 10⁸-10¹⁰, or 10⁸-10⁹ TU/ml).

Transduced NK cells are produced by contacting the activated NK cells described in Section IIB with the lentiviral particles, for example at a multiplicity of infection (MOI) of about 0.5 to 200 (such as about 0.5-5, 1-10, 5-15, 10-25, 20-50, 40-80, 60-100, 75-150, or 100-200, for example, about 0.5, 1, 2, 5, 10, 15, 20, 25, 30, 40, 50, 60, 70, 80, 90, 100, 125, 150, 175, or 200). In some examples, the NK cells are contacted with virus at an MOI of about 10-20 or an MOI of about 20. In some examples, the transduction is in the presence of one or more additional compounds, such as protamine sulfate (for example, 5-50 μg/ml, such as 5, 10, 20, 30, 40, or 50 μg/ml) or hexadimethrine bromide (e.g., Polybrene®, for example about 4-40 μg/ml, such as 4, 8, 12, 16, 20, 24, 28, 32, 36, or 40 μg/ml), or a fibronectin fragment (e.g., Retronectin®). The cells are cultured with the viral particles for about 6 hours to 5 days (for example, about 6-24 hours, 12-48 hours, 24-72 hours, 48-60 hours, or 72-96 hours), such as about 1, 2, 3, 4, or 5 days.

In some embodiments, the NK cells are transduced in the presence of one or more inhibitors of innate immune signaling or viral sensing, such as an inhibitor of toll-like receptor (TLR) and/or RIG-I-like receptor (RLR), an inhibitor of TBK1/IKKε, an inhibitor of AIM2-like receptors (ALR), an inhibitor of cyclic-GMP-AMP (cGAS), and/or an inhibitor of stimulator of interferon genes (STING). Exemplary inhibitors of TLR, RLR, ALR, cGAS, and/or STING include ST2825, chloroquine, hydroxychloroquine, CpG-52364, SM934, IRS-954, DV-1179, IMO-3100, IMO-8400, IMO-9200, IHN-ODN 2088, IHN-ODN-24888, RU.521, PF-06928215, Pepinh-MYD, Pepinh-TRIF, Z-VAD-FMK, 2-aminopurine, BAY11-7082, Celastrol, CLI-095, H-89, Norharmane, and IRAK1/4 inhibitor (see e.g., Gao et al., Front. Physiol. 8:508, 2017; Vincent et al., Nat. Commun. 8:750, 2017; Sutlu et al., Human Gene Ther. 23:1090-1100, 2012; Hall et al., PLOS One 12:e0184843, 2017). Exemplary inhibitors of TBK1/IKKε include BX795, MRT67307, AZ13102909, MPI-0485520, Amlexanox, Compound II, SR8185 and CYT387 (see, e.g., Hasan et al., Pharmacol. Res. 111:336-342, 2016). In some examples, the NK cells are transduced in medium including the inhibitor in the presence or absence of feeder cells. In one non-limiting example, the inhibitor is BX795, such as about 0.25 μM to 15μ M (for example, about 0.25μ M to about 3μ, about 0.5μ M to about 5μ, about 1.5μ M to about 12 μM, about 0.75 μM to about 3 μM, or about 0.5 μM, about 0.75 μM, about 1.5 μM, about 3 μM, about 6 μM, or about 12 μM). In one non-limiting example, the inhibitor is 1.5 μM BX795.

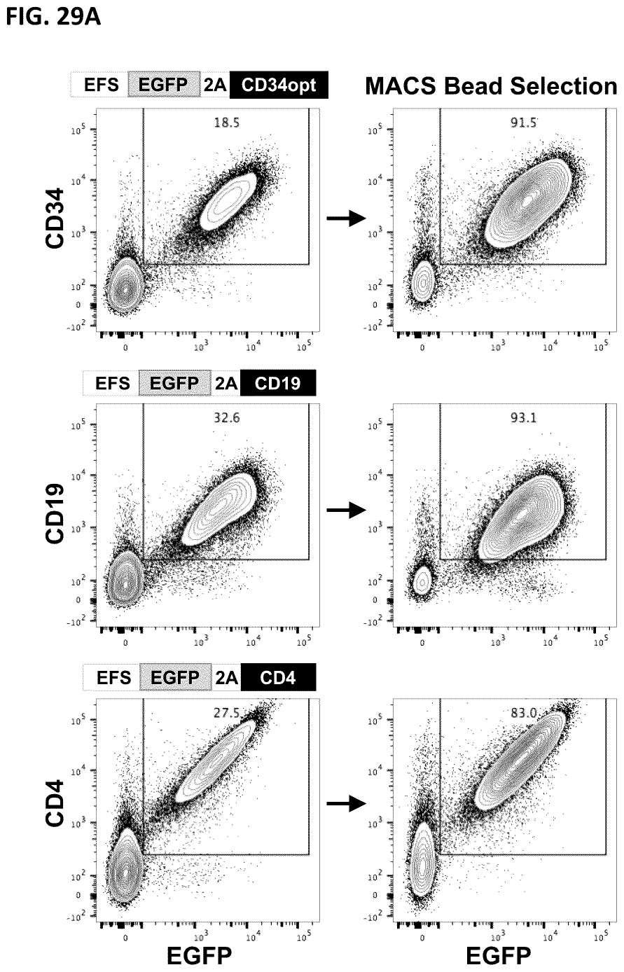

Following transduction of the NK cells with the lentivirus, the viral particles (and one or more inhibitors of innate immune signaling or viral sensing, if present) are removed (for example by exchanging the culture medium and optionally washing the cells). In some examples, NK cells expressing the transgene are optionally selected prior to expansion (below). For example, if the transgene is expressed on the cell surface, NK cells expressing the transgene may be enriched by immuno-magnetic techniques or flow cytometry. For example, if the NK cells are transduced with a vector including a nucleic acid encoding a truncated CD34 molecule (CD34t), a truncated CD19 molecule, or a truncated CD4 molecule, transduced NK cells can be selected or enriched by contacting the population of transduced NK cells with an appropriate antibody (e.g., anti-CD34 antibody) and purifying NK cells expressing the molecule (e.g., CD34) for example, using flow cytometry or immuno-magnetic beads (e.g., CliniMACS® CD34 reagent system, Miltenyi Biotec Inc., San Diego, CA or Isolex® 300 magnetic cell selection system, Nexell Therapeutics Inc., Irvine, CA), for example about 2-4 days after transduction. NK cells expressing other transgenes (such as CD19 or CD4) can be selected or enriched similarly, using flow cytometry or immuno-magnetic beads specific for the transgene (see, e.g., FIG. 29A).

The transduced NK cells are then expanded by culturing the cells for 1-40 days or more (such as 3-14 days, 5-21 days, 7-28 days, 14-30 days, 21-40 days, or more, for example, 1, 3, 5, 7, 10, 14, 21, 28, 35, 42 days, or more). In some examples, the transduced NK cells are expanded in cell culture medium containing at least one cytokine. In some examples, the transduced NK cells are expanded by culturing in a medium (such as X-VIVO™ 20 or X-VIVO™ 15 medium (Lonza, Basel, Switzerland)) including IL-2 (such as 1-1000 IU/ml, for example, 500 IU/ml IL-2). In some embodiments, one or more additional cytokines can be utilized in the expansion of the transduced NK cells, including but not limited to IL-18, IL-7, IL-15, and/or IL-12. In other examples, the population of transduced NK cells is cultured in the presence of IL-2 and no other added cytokines.

In some examples, feeder cells (such as irradiated feeder cells) are added to the transduced NK cell culture during the expansion step. The feeder cells are added in an amount that can support expansion of the NK cells, such as at least 2:1 ratio (feeder cells:NK cells), for example, 5:1, 10:1, 15:1, 20:1, or more. Exemplary feeder cells include EBV-LCLs (TM-LCL, SMI-LCL), allogeneic or autologous PBMCs, Wilms tumor cell line HFWT, and K562 cells (such as genetically modified K562 cells, for example, K562-mb15-41BBL or K562-mbIL-21 cells). In some examples, the transduced NK cells are mixed with the feeder cells at the selected ratio and the cell mixture is plated.

Utilizing these techniques, expansion of the transduced NK cells in the range of 100- to 1000-fold (such as 200- to 500-fold, 300- to 700-fold, or 600- to 1000-fold) may be achieved.

Following expansion, the modified NK cells are separated from the feeder cells (if used). The modified NK cells are washed one or more times and resuspended in an appropriate buffer or other pharmaceutically acceptable carrier, for example, for administration to a subject. In some examples, the cells are harvested and washed (for example in a buffer, such as phosphate buffered saline). The NK cells may be resuspended in a medium containing PLASMA-LYTE™ multiple electrolytes injection (Baxter Healthcare), autologous plasma, or a pharmaceutically acceptable carrier (for example, a balanced salt solution). In some examples, some or all of the modified NK cells are cryopreserved for later use.

In some examples, the modified NK cells are tested prior to administering to a subject, for example, for one or more of cell viability, tumor cell cytotoxicity, and transgene expression. In additional examples, the phenotype of the modified NK cells is assessed prior to administration, such as by measuring presence and/or amount of one or more cell surface markers (such as CD56, CD16, TRAIL, FasL, NKG2D, LFA-1, perforin, or granzymes A and B), for example by flow cytometry.

### III. Modified NK Cells

Disclosed herein are NK cells including a heterologous nucleic acid or expressing one or more heterologous proteins or a nucleic acid of interest (referred to herein as “modified NK cells”). Modified or recombinant NK cells containing one or more heterologous nucleic acids can be produced by transducing NK cells with virus particles including a vector (such as a lentivirus vector) with the one or more heterologous nucleic acids, for example using the methods disclosed herein. The modified NK cells can be formulated into a therapeutic composition for administration to a subject, for example, with one or more pharmaceutically acceptable carriers. Therapeutic compositions and methods of their use are discussed in Section IV, below.

The modified NK cells are NK cells that have been transduced with a viral vector or virus particle (such as a lentiviral vector or lentivirus particle) including one or more transgenes, such as one or more heterologous nucleic acids encoding a protein of interest or capable of regulating expression of another nucleic acid or protein (such as an shRNA, siRNA, or antisense nucleic acid). In some embodiments, the nucleic acid encodes a protein that facilitates targeting of the modified NK cells expressing the protein to a target tissue or cell type. For example, the nucleic acid can encode a protein that increases targeting of an NK cell expressing the protein to tumor cells or sites of tumor cells. In one non-limiting example, the transgene increases targeting of NK cells to bone marrow or lymph nodes (for example, C-C chemokine receptor type 7 (CCR7), C-X-C chemokine receptor type 4 (CXCR4; e.g., wild type CXCR4 or CXCR4 R334X), or CD34) or sites of inflammation (such as C-X-C chemokine receptor type 3 (CXCR3)). In other examples, the transgene encodes a protein that binds to an epitope expressed on tumor cells (such as a chimeric antigen receptor (CAR)). In other embodiments, the nucleic acid encodes a protein that increases antibody-dependent cell mediate cytotoxicity of NK cells, such as high affinity CD16 (CD16-V158).

Thus, in particular examples, the modified NK cells disclosed herein are NK cells (such as a population of NK cells) that include a heterologous nucleic acid encoding a chemokine receptor, such as CXCR4, CCR7, or CXCR3. In other examples, the modified NK cells are NK cells (such as a population of NK cells) that include a heterologous nucleic acid encoding a cell surface protein, such as CD16 (for example, CD16-V158), CD34, double negative TGFβ type II receptor, VLA-4 (alpha4beta-1), LFA-1, CD4, or CD19. In additional examples, the modified NK cells are NK cells (such as a population of NK cells) that include a heterologous nucleic acid encoding a chimeric antigen receptor (CAR), for example CD19-CAR, CD20-CAR, CD33-CAR, CD138-CAR, CS1-CAR, GD2-CAR, HER2-CAR, erbB2-CAR, carcinoembryonic antigen (CEA)-CAR, epithelial cell adhesion molecule (EpCAM)-CAR, natural-killer group 2, member D, long form (NKG2D-L)-CAR, or TRAIL receptor 1 (TRAIL-R1)-CAR. In still further examples, the modified NK cells are transduced with recombinant TRAIL (e.g., to enhance NK cell TRAIL-mediated tumor killing) DR5-specific TRAIL, recombinant FAS-Ligand (e.g. to enhance FAS-Ligand mediated tumor killing), DNAM-1 (e.g., to enhance NK cell activation and tumor killing), NK cell activating receptors such as NKG2D, DNAM-1, NKp30, NKp44, or NKp46 (e.g., to enhance tumor killing), or NKG2C (e.g., to enhance NK cell killing of viral infected cells).

In one non-limiting example, the modified NK cells include a heterologous nucleic acid encoding CXCR4 operably linked to a promoter (such as a PGK promoter) or nucleic acids encoding CD34t and CXCR4 operably linked to a promoter (such as a PGK promoter). In other non-limiting examples, the modified NK cells include a heterologous nucleic acid encoding high affinity CD16 operably linked to a promoter (such as a PGK promoter) or nucleic acids encoding CD34t and high affinity CD16 operably linked to a promoter (such as a PGK promoter). In another non-limiting example, the modified NK cells include a heterologous nucleic acids encoding CXCR4 and high affinity CD16 operably linked to a promoter (such as a PGK promoter) or nucleic acids encoding CD34t, CXCR4, and high affinity CD16 operably linked to a promoter (such as a PGK promoter). In some examples, the nucleic acid encoding CXCR4 includes or consists of a nucleic acid with at least 90% identity (such as at least 90%, 91%, 92%, 93%, 94%, 95%, 96%, 97%, 98%, 99%, or 100% identity) to SEQ ID NO: 1 and/or encodes a protein including or consisting of an amino acid sequence with at least 95% identity (such as at least 95%, 96%, 97%, 98%, 99%, or 100% identity) to SEQ ID NO: 2. In some examples, the nucleic acid encoding CD34t includes or consists of a nucleic acid with at least 90% identity (such as at least 90%, 91%, 92%, 93%, 94%, 95%, 96%, 97%, 98%, 99%, or 100% identity) to SEQ ID NO: 3 and/or encodes a protein including or consisting of an amino acid sequence with at least 95% identity (such as at least 95%, 96%, 97%, 98%, 99%, or 100% identity) to SEQ ID NO: 4. In some examples, the nucleic acid encoding high affinity CD16 includes or consists of a nucleic acid with at least 90% identity (such as at least 90%, 91%, 92%, 93%, 94%, 95%, 96%, 97%, 98%, 99%, or 100% identity) to SEQ ID NO: 5 or SEQ ID NO: 6 and/or encodes a protein including or consisting of an amino acid sequence with at least 95% identity (such as at least 95%, 96%, 97%, 98%, 99%, or 100% identity) to SEQ ID NO: 7.

In other examples, the modified NK cells can be transduced with a viral vector including a nucleic acid coding for small interfering RNAs (siRNAs), such as small hairpin RNA (shRNA). In one example, the modified NK cells are NK cells (such as a population of NK cells) that include a heterologous NKG2A shRNA nucleic acid.

### IV. Methods of Treating or Inhibiting a Condition

Disclosed herein are methods of treating a subject with a disease or disorder by administering the modified NK cells described herein to the subject. The modified NK cells described herein can be administered either to animals or to human subjects. In some embodiments, the disease or disorder is a tumor or hyperproliferative disease. In other embodiments, the disease or disorder is a viral infection (including but not limited to cytomegalovirus, adenovirus, respiratory syncytial virus, Epstein-Barr virus, or human immunodeficiency virus infection).

The modified NK cells described herein can be incorporated into pharmaceutical compositions. Such compositions typically include a population of modified NK cells and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington: The Science and Practice of Pharmacy, The University of the Sciences in Philadelphia, Editor, Lippincott, Williams, & Wilkins, Philadelphia, PA, 21^{st} Edition, 2005). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions are known or apparent to those skilled in the art and are described in more detail in such publications as Remington: The Science and Practice of Pharmacy, The University of the Sciences in Philadelphia, Editor, Lippincott, Williams, & Wilkins, Philadelphia, PA, 21^{st} Edition (2005). In one non-limiting example, the transduced NK cells are suspended in PLASMA-LYTE™ multiple electrolyte solution.

In some examples, the composition includes about 10⁴ to 10¹² of the modified NK cells (for example, about 10⁴-10⁷ cells, about 10⁶-10⁹ cells, or about 10⁸-10¹² cells). For example, the composition may be prepared such that about 10⁶ to 10¹⁰ modified NK cells/kg (such as about 10⁶, 10⁷, 10⁸, 10⁹, or 10¹⁰ cells/kg) are administered to a subject. The population of modified NK cells is typically administered parenterally, for example intravenously; however, injection or infusion to a tumor or close to a tumor (local administration) or administration to the peritoneal cavity can also be used. One of skill in the art can determine appropriate routes of administration.

Multiple doses of the population of modified NK cells can be administered to a subject. For example, the population of modified NK cells can be administered daily, every other day, twice per week, weekly, every other week, every three weeks, monthly, or less frequently. A skilled clinician can select an administration schedule based on the subject, the condition being treated, the previous treatment history, and other factors.

In additional examples, the subject is also administered one or more cytokines (such as IL-2, IL-15, IL-21, and/or IL-12) to support survival and/or growth of NK cells. The cytokine(s) are administered before, after, or substantially simultaneously with the NK cells. In some examples, the cytokine(s) are administered after the NK cells. In one specific example, the cytokine(s) is administered to the subject within about 1-8 hours (such as within about 1-4 hours, about 2-6 hours, about 4-6 hours, or about 5-8 hours) after administration of the NK cells.

In some examples, the methods include treating or inhibiting a hyperproliferative disorder, such as a hematological malignancy or a solid tumor. Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, diffuse large B-cell lymphoma, Hodgkin's lymphoma, non-Hodgkin's lymphoma (indolent and high grade forms), mantle cell lymphoma, follicular cell lymphoma, multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma).

In particular examples, hematological malignancies that can be inhibited or treated by the methods disclosed herein include but are not limited to multiple myeloma, chronic lymphocytic leukemia, acute lymphocytic leukemia, acute myeloid leukemia, chronic myelogenous leukemia, pro-lymphocytic/myelocytic leukemia, plasma cell leukemia, NK cell leukemia, Waldenstrom macroglobulinemia, Hodgkin's lymphoma, non-Hodgkin's lymphoma, mantle cell lymphoma, diffuse large B-cell lymphoma, and follicular lymphoma. In additional particular examples, solid tumors that can be treated or inhibited by the methods disclosed herein include lung carcinoma, prostate cancer, pancreatic cancer (for example, insulinoma), breast cancer, colorectal adenocarcinoma or squamous cell carcinoma, neuroblastoma, testicular cancer (such as seminoma), and ovarian cancer. In specific, non-limiting examples, the subject has chronic myelogenous leukemia or acute monocytic leukemia. Exemplary transgenes for expression by modified NK cells that can be used for treating a subject with exemplary disorders are shown in Table 1. However, one of ordinary skill in the art can select NK cells expressing an appropriate transgene for treating a subject with other disorders.

TABLE 1
Exemplary modified NK cells for treating particular disorders

| Transgene Expressed by | |
|---|---|
| Modified NK Cells | Disorder |
| CD16 V158 | Multiple tumor types, including |
| | multiple myeloma and lymphoma |
| CCR7 | Lymphoma |
| CXCR3 | Tumor metastases |
| CXCR4 | Breast cancer, kidney cancer, |
| | multiple myeloma |
| CD34 | Leukemia |
| CD19-CAR | B cell malignancies |
| CD20-CAR | B cell malignancies |
| CD33-CAR | leukemia |
| CS1-CAR | Myeloma |
| CD138-CAR | Myeloma |
| GD2-CAR | Neuroblastoma, melanoma |
| Her2/Neu-CAR | Breast cancer |
| ErbB2-CAR | Breast cancer |
| CEA-CAR | Colon cancer |
| EpCAM-CAR | Epithelial tumors |
| NKG2D-CAR | Leukemia, solid tumors |
| TRAIL-R1-CAR | Multiple tumor types |
| DNTβRII | Lung cancer |
| TRAIL | Multiple tumor types |
| FAS-Ligand | Multiple tumor types |
| DNAM-1 | Multiple tumor types |
| NKG2D | Multiple tumor types |
| NKp30 | Multiple tumor types |
| NKp44 | Multiple tumor types |
| NKp46 | Multiple tumor types |
| NKG2C | Viral infection |
| NKG2A shRNA | Leukemia |

In some examples, the subject (such as a subject with a tumor or hyperproliferative disorder) is also administered one or more chemotherapeutic agents and/or radiation therapy. One of skill in the art can select additional chemotherapeutic agents for administration to a subject in combination with the modified NK cells described herein, for example, based on the type of tumor or disorder being treated). Such agents include alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol.

In some particular examples, the modified NK cells express CD16 V158 and the additional agent is an anti-cancer monoclonal antibody. In specific, non-limiting examples, modified NK cells expressing CD16-V158 are administered to a subject with multiple myeloma in combination with an antibody that binds to CD38 (such as daratumumab). In another particular example, modified NK cells expressing CD16-V158 are administered to a subject with lymphoma in combination with an antibody that binds to CD20 (such as rituximab). Additional exemplary monoclonal antibodies that can be administered to a subject in combination with modified NK cells expressing CD16-V158 are provided in Table 2.

TABLE 2
Exemplary therapeutic monoclonal antibodies for administration
in combination with NK cells expressing CD16-V158

| Antigen | mAb | Target Tumor/Disease |
|---|---|---|
| CD19 | GBR 401, MEDI-551 | B cell lymphoma, CLL |
| CD20 | Rituximab (RITUXAN ®), | Non-Hodgkin's lymphoma |
| | ofatumumab (ARZERRA ®), | |
| | veltuzumab | |
| | Ibritumomab tiuxetan | Lymphoma |
| | (ZEVALIN ®), obinutuzumab, | |
| | ublituximab, tositumomab | |
| | (BEXXAR ®), ocaratuzumab | |
| CD22 | Narnatumab, inotuzumab | Cancer |
| | ozogamicin | |
| CD30 | Brentuximab vedotin | Hodgkin's lymphoma |
| | (ADCETRIS ®), iratumumab | |
| CD33 | Gemtuzumab ozogamicin | Acute myelogenous leukemia |
| | (MYLOTARG ®), lintuzumab, | |
| CD37 | Otlertuzumab | Cancer cells |
| CD38 | Daratumumab | Multiple myeloma |
| CD40 | Lucatumumab, | multiple myeloma, non-Hodgkin's |
| | dacetuzumab | or Hodgkin's lymphoma |
| CD52 | Alemtuzumab (CAMPATH ®, | Chronic lymphocytic |
| | MABCAMPATH ®, | leukemia |
| | CAMPATH-1H ®) | |
| CD56 | Lorvotuzumab mertansine | small-cell lung cancer, ovarian |
| | | cancer |
| CD70 | Vorsetuzumab mafodotin | Renal cell carcinoma |
| CD74 | Milatuzumab | Multiple myeloma |
| CD140 | Tovetumab | cancer |
| EpCAM | IGN101, oportuzumab | Epithelial tumors (breast, colon |
| | monatox, tucotuzumab | and lung) |
| | celmoleukin, adecatumumab | |
| CEA | Labetuzumab (CEA-CIDE ®) | Breast, colon and lung tumors |
| gpA33 | huA33 | Colorectal carcinoma |
| mesothelin | Amatuximab | Cancer cells |
| α-fetoprotein | ⁹⁰Y-tacatuzumab | Tumor cells |
| | tetraxetan | |
| IL-6 | Siltuximab | metastatic renal cell cancer, |
| | | prostate cancer, and Castleman's |
| | | disease |
| Mucins | Pemtumomab (THERAGYN ®), | Breast, colon, lung and ovarian |
| | cantuzumab mertansine, ⁹⁰Y | tumors |
| | clivatuzumab tetraxetanand, | |
| | oregovomab (OVAREX ®) | |
| PDGFR-alpha | Olaratumab | Solid tumors |
| TAG-72 | CC49 (minretumomab) | Breast, colon and lung tumors |
| CAIX | Girentuximab, cG250 | Renal cell carcinoma |
| PSMA | J591 | Prostate carcinoma |
| Folate-binding | MOv18 and MORAb-003 | Ovarian tumors |
| protein | (farletuzumab) | |
| Scatter factor | Onartuzumab | Cancer cells |
| receptor kinase | | |
| Gangliosides | 3F8, ch14.18, KW-2871 | Neuroectodermal |
| (e.g., GD2, | | tumors and some |
| GD3 and GM2) | | epithelial tumors |
| Cytokeratin | ^{99m}Tc- Votumumab | Colorectal tumors |
| | (HUMASPECT ®) | |
| Frizzled | Vantictumab | cancer |
| receptor | | |
| Le^{y} | hu3S193, IgN311 | Breast, colon, lung and |
| | | prostate tumors |
| VEGF | Bevacizumab (AVASTIN ®) | Tumor vasculature |
| VEGFR | IM-2C6, CDP791 | Epithelium-derived solid |
| | | tumors |
| Integrin | Etaracizumab (ABEGRIN ®), | Tumor vasculature |
| αVβ3 | intetumumab | |
| Integrin α5β1 | Volociximab | Tumor vasculature |
| EGFR | Cetuximab (ERBITUX ®), | Glioma, lung, breast, |
| | panitumumab (VECTIBIX ®), | colon, and head and |
| | nimotuzumab, necitumumab, | neck tumors |
| | zalutumumab, imgatuzumab, | |
| | matuzumab, 806 | |
| EGFL7 | Parsatuzumab | Cancer cells |
| ERBB2 | Trastuzumab (HERCLON ®; | Breast, colon, lung, |
| | HERCEPTIN ®), pertuzumab | ovarian and prostate |
| | (PERJETA ®; OMNITARG ®) | tumors |
| ERBB3 | Duligotumab, MM-121 | Breast, colon, lung, |
| | | ovarian and prostate, |
| | | tumors |
| Fibronectin | Radretumab | antineoplastic |
| HGF | Rilotumumab, ficlatuzumab | Solid tumors |
| HER3 | Patritumab | cancer |
| LOXL2 | Simtuzumab | fibrosis |
| MET | AMG 102, METMAB, SCH | Breast, ovary and lung |
| | 900105 | tumors |
| IGF1R | Cixutumumab, dalotuzumab, | Glioma, lung, breast, |
| | figitumumab, ganitumab, | head and neck, |
| | robatumumab, teprotumumab, | prostate and thyroid |
| | AVE1642, IMC-A12, MK-0646, | cancer |
| | R1507, and CP 751871 | |
| IGLF2 | Dusigitumab | |
| EPHA3 | KB004, IIIA4 | Lung, kidney and colon |
| | | tumors, melanoma, glioma |
| | | and hematological malignancies |
| FR-alpha | Farletuzumab | Ovarian cancer |
| phosphatidylserine | Bavituximab | Cancer cells |
| Syndecan 1 | Indatuximab ravtansine | |
| SLAMF7 (CD319) | Elotuzumab | Multiple myeloma |
| TRAILR1 | Mapatumumab (HGS-ETR1) | Colon, lung and pancreas |
| | | tumors and hematological |
| | | malignancies |
| TRAILR2 | Conatumumab, lexatumumab, | cancer |
| | mapatumumab, tigatuzumab, | |
| | HGS-ETR2, CS-1008 | |
| RANKL | Denosumab (XGEVA ®) | Prostate cancer and bone |
| | | metastases |
| FAP | Sibrotuzumab, and F19 | Colon, breast, lung, |
| | | pancreas, and head and |
| | | neck tumors |
| vimentin | Pritumumab | Brain cancer |
| Tenascin | 81C6 | Glioma, breast and |
| | | prostate tumors |

### EXAMPLES

The following examples are provided to illustrate certain particular features and/or embodiments. These examples should not be construed to limit the disclosure to the particular features or embodiments described.

### Example 1

### Materials and Methods

Cell Lines and Reagents: The human myelogenous leukemia line (erythroleukemia type) K562 (ATCC) and the acute monocytic leukemia cell line MOLM-14 (ATCC), and EBV-SMI-LCL were cultured in RPMI 1640 supplemented with 10% heat-inactivated fetal bovine serum (FBS, Sigma-Aldrich), 2 mM glutamine, and 100 U/mL penicillin, and 100 μg/mL streptomycin (Life Technologies). The 293T cell line, human embryonic kidney cell line 293 stably expressing SV40 large T antigen (from ATCC) was cultured in Dulbecco's modified Eagle's medium (DMEM; Invitrogen) supplemented with 2 mM L-glutamine, 100 U/mL penicillin, and 100 μg/mL streptomycin (Invitrogen) and 10% FBS. The cells were cultured under 95% humidity at 37° C. and 5% CO₂.

Culture and expansion of human peripheral blood-derived NK cells: Peripheral blood mononuclear cells (PBMC) from healthy donors were collected by apheresis (Amicus Separator; Fenwal, Lake Zurich, IL, USA) on an institutional review board-approved protocol in the Department of Transfusion Medicine, National Institutes of Health (Bethesda, MD, USA). Cells were enriched by centrifugation over Ficoll density gradient medium. NK cells were isolated from donor PBMCs using the NK cell isolation kit from Miltenyi following the manufacturer's instructions. Where indicated NK cells were expanded for 11-21 days in NK cell media (X-VIVO™ 20 medium (Lonza) supplemented with 10% heat-inactivated human AB plasma (Sigma-Aldrich) and 500 IU/ml of recombinant human IL-2 (Roche)) in the presence of irradiated EBV-SMI-LCL cells at a ratio of 1:10. The cells were cultured at 37° C. and 6.5% CO₂. Half of the media was replaced with fresh NK cell media 5 days into the expansion. Thereafter, NK cells were counted and adjusted to 0.5-1×10⁶ cells/ml every 48 h, from day 7 until utilized in experiments.

Lentiviral vector system: The human immunodeficiency virus (HIV)-1-based lentiviral gene transfer vector used in this study was similar to a self-inactivating construct pRRL-CMV-eGFP-SIN-18 described by Dull et al. (J Virol. 72:8463-8471, 1998) except that an additional 118-bp sequence containing the central poly purine tract (cPPT) had been introduced in the vector. In addition, the woodchuck hepatitis virus post-transcriptional regulatory element (WPRE) backbone was included to augment transgene expression and/or viral vector titer. The internal cytomegalovirus (CMV) early promoter CMV promoter used to drive the transcription of transgene was replaced with the human phosphoglycerate kinase (PGK) promoter by polymerase chain reaction (PCR)-based amplification. The packaging plasmid pCMVΔR8.91 (devoid of all HIV-1 accessory genes) was used to express the HIV-1 gag, pol, tat, and rev genes and thereby produce lentiviral structural and regulatory proteins. The plasmid pMD.G carrying the vesicular stomatitis virus envelope G protein (VSV-G) coding sequence driven by the CMV promoter and followed by the β-globin polyadenylation site was used to pseudotype the vector particles. The plasmids pRRL-CMV-eGFP-SIN-18, pCMVΔR8.91, and pMD.G were kindly provided by Prof. D. Trono, Department of Genetics and Microbiology, CMU, Geneva, Switzerland.

Lentiviral vector production: Replication-defective lentiviral particles pseudotyped with VSV-G envelope were produced by 3-plasmid transient transfection of 293T cells with 12 μg of the gene transfer construct pRRLsin.PPT.hPGK.eGFP.Wpre, 10 μg of pCMVΔR8.91, and 5 μg of pMD.G, using a calcium phosphate transfection kit (Clontech) as described previously (Chinnasamy et al., Blood 96:1309-1316. 2000). The transfection medium was replaced after 8 hours with fresh culture medium. Viral supernatants were harvested at 65 hours after transfection and filtered through 0.45 μm filters (Nalgene, Rochester, NY). The viral supernatants were concentrated to 50× by ultracentrifugation at 50,000 g for 1.5 hours at 4° C.). Viral pellets were resuspended in X-VIVO™ 20 medium (Lonza) and stored frozen at −80° C. until use. Titers of viral supernatants were determined by quantification of p24 gag by enzyme-linked immunosorbent assay (ELISA) (Coulter Diagnostics, Hialeah, FL), and also by transducing 293T cells with serial dilutions of viral supernatants, followed by flow cytometry analysis of EGFP-positive cells, 48 hours after transduction. It was assumed that 1 ng of p24 is approximately equivalent to 1000 to 5000 transducing units. All lentiviral vector preparations were tested for the presence of replication-competent lentivirus (RCL) by transducing 293T cells and assaying culture medium for the presence of p24 gag after at least 5 cell passages. No RCL was detectable in any of the vector preparations tested.

Lentiviral transduction of primary human NK cells primed with cytokines or stimulated with LCL: Cultured human NK cells were transduced with concentrated lentiviral vector particles at a multiplicity of infection (MOI), in the presence of protamine sulfate (10 μg/ml, Sigma-Aldrich), in 24-well plate containing 1 ml/well of NK culture medium supplemented with IL-2 (500 IU/ml or 1000 IU/ml), IL-15 (10 ng/ml), or IL-2 (500 IU/ml) and IL15 (10 ng/ml). After 48 hours transduction, cells were washed and cultured in fresh NK cell culture medium containing IL-2 (500 IU/ml) in the presence or absence of irradiated EBV-SMI-LCL (LCL to NK ratio of 10:1). In some conditions, primary NK cells were co-cultured with EBV-SMI-LCL (LCL to NK ratio of 10:1) at different lengths of time prior to transduction with lentiviral vector for 48 hours and then cells were washed and cultured in fresh NK cell culture medium containing IL-2 (500 IU/ml). The viability of NK cultures was monitored periodically before and after transduction using trypan blue (Sigma) staining. The phenotype and eGFP expression were monitored at regular intervals by flow cytometry.

Flow cytometry: The cultured NK cells were phenotyped by flow cytometry at different time points post culture and expansion according to standard procedures. The following reagents were used in phenotypic characterization: anti-CD56 (NCAM-1), anti-CD3 (UCHT1), anti-CD16 (3G8), anti-TRAIL (RIK-2), anti-NKp46 (29A1.4), and IgG1 (MOPC21) from Becton Dickinson (BD); anti-KIR2DL1/DS1 (EB6), anti-KIR2DL2/3/DS2 (GL183), and anti-NKG2A (Z199) from Beckman Coulter; the anti-KIR3DL1 (Dx9), anti-CD57 (HCD57), NKp44 (Clone p44-8, FAS (clone DX2), CXCR3 (clone 12G5), CXCR4 (clone G025H7), and BV650-streptavidin from Biolegend; the anti-Lir-1 (HP-F1) from eBioscience; the anti-NKG2C (134591) from R&D Systems; LIVE/DEAD viability marker or propidium iodide (PI) from Life Technologies; biotinylated anti-KIR3DL2 (Dx31) primary antibody from UCSF. The BV650-streptavidin (Biolegend) was used to detect the biotinylated anti-KIR3DL2. Briefly, cells were mixed with appropriate concentrations of different dye-conjugated monoclonal antibodies (mAbs). After the addition of primary Ab and incubation for 20 minutes at room temperature, cells were washed with PBS containing 1% FBS. Propidium iodide (PI) or LIVE/DEAD staining was used for dead cell exclusion. All the data were acquired using the Canto II or Fortessa flow cytometer with FACS Diva software (BD Biosciences) and analyzed using the FlowJo software (Tree Star). In each sample, a minimum of 10,000 cells was acquired in the analysis region of viable cells, using log-amplified fluorescence and linearly amplified side- and forward-scatter signals. All samples were analyzed by setting appropriate gates around the lymphocyte population, using LIVE/DEAD or PI-negative cells. Consistency of analysis parameters was ascertained by calibrating the flow cytometer with calibrating beads (BD biosciences). EGFP expression in transduced cells was determined by detecting the % green fluorescence positive cells using a 530/30 nm band pass filter in the FL1 channel.

Cytotoxicity Assay: Natural killer cells were co-cultured at a ratio of 1:1 with either ⁵¹Cr-labeled K562 cells or Molm-14 cells in a final volume of 200 μl in 96-well plates at 37° C. and 5% CO₂. After 4 hours, supernatant was harvested onto a Luma plate. Counts were measured using a Perkin Elmer 1450 Microbeta Counter and specific target lysis was calculated using the following formula: [(NK cell-induced ⁵¹Cr release−spontaneous ⁵¹Cr release)/(maximum ⁵¹Cr release−spontaneous ⁵¹Cr release)×100].

### Example 2

### Effect of IL-2 Priming on Lentiviral Transduction of NK Cells

This example describes the effect of priming with IL-2 on transduction of NK cells using a lentiviral vector and characterization of the transduced NK cells.

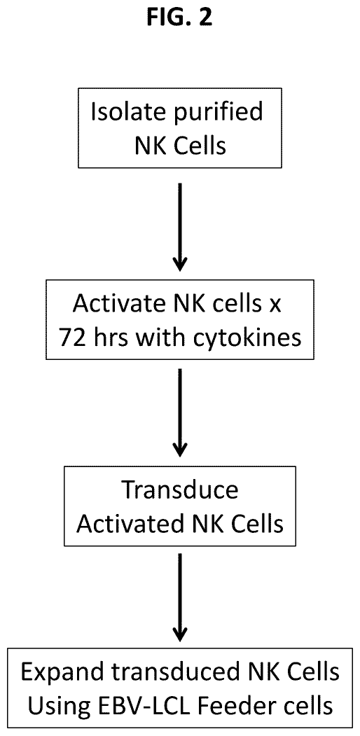

Conditions for priming NK cells prior to transduction and culture conditions post-transduction were evaluated. In initial experiments, priming of NK cells with IL-2 was evaluated. In one experiment, NK cells on LCL feeder cells were primed with 500 IU/ml IL-2 for three days prior to transduction with various multiplicity of infection (MOI) of lentiviral particles including an eGFP transgene. Cells were incubated with viral particles for two days, then viral particles were washed off and the cells were cultured with media including 500 IU/ml IL-2. In a second experiment, NK cells were primed with 500 IU/ml IL-2 for three days prior to transduction with lentiviral particles including the eGFP transgene. Cells were incubated for two days, then viral particles were washed off and the cells were cultured on irradiated LCL feeder cells with media including 500 IU/ml IL-2. eGFP expression was evaluated by FACS on day 7 post-transduction. eGFP expression on CD56+ cells was similar between the two conditions (FIG. 3), but was slightly higher in the cells primed with LCL and IL-2 and grown on IL-2 only post-transduction (Condition 1).

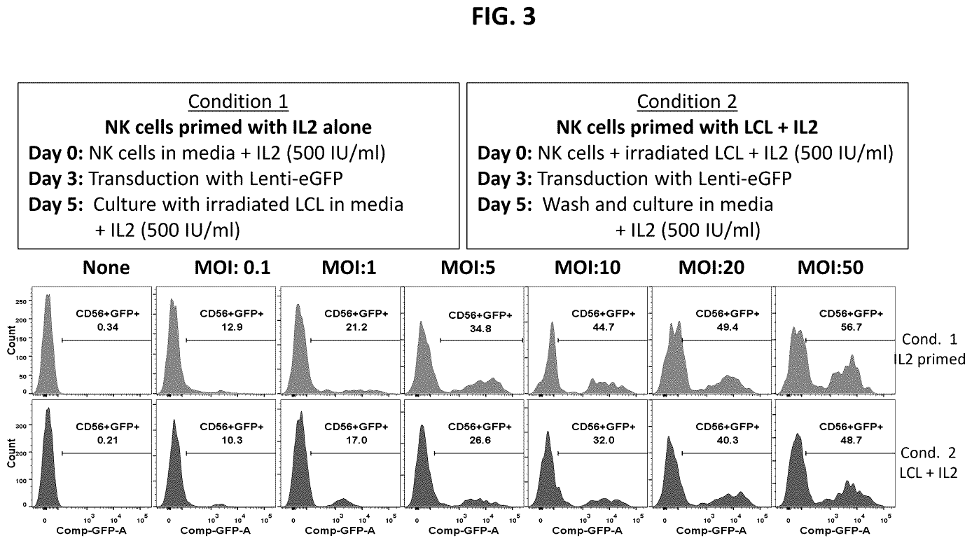

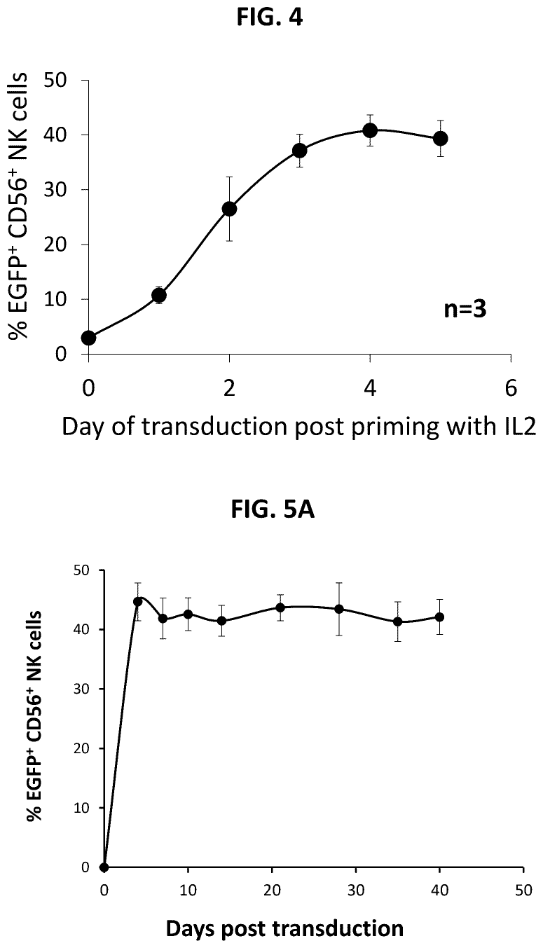

The impact of IL-2 priming on NK cell transduction was evaluated by priming NK cells with 500 IU/ml IL-2 for 1-5 days prior to transduction. At two days post-transduction, viral particles were removed and irradiated LCLs were added at a 10:1 ratio (LCL:NK cells) and the cells were maintained in medium containing 500 IU/ml IL-2. Priming with IL-2 for two or more days resulted in transduction efficiency of about 25% or more (FIG. 4). NK cells were also primed with medium containing IL-2 (500 IU/ml) for three days prior to transduction with at MOI 20. Two days post-transduction, viral particles were removed, irradiated LCLs (10:1 ratio LCL:NK cells) were added, and the cells were maintained in medium containing 500 IU/ml IL-2. Transgene expression remained stable for at least 40 days post-transduction (FIG. 5A) and cell viability remained comparable to untransduced cells for at least 28 days post-transduction (FIG. 5B).

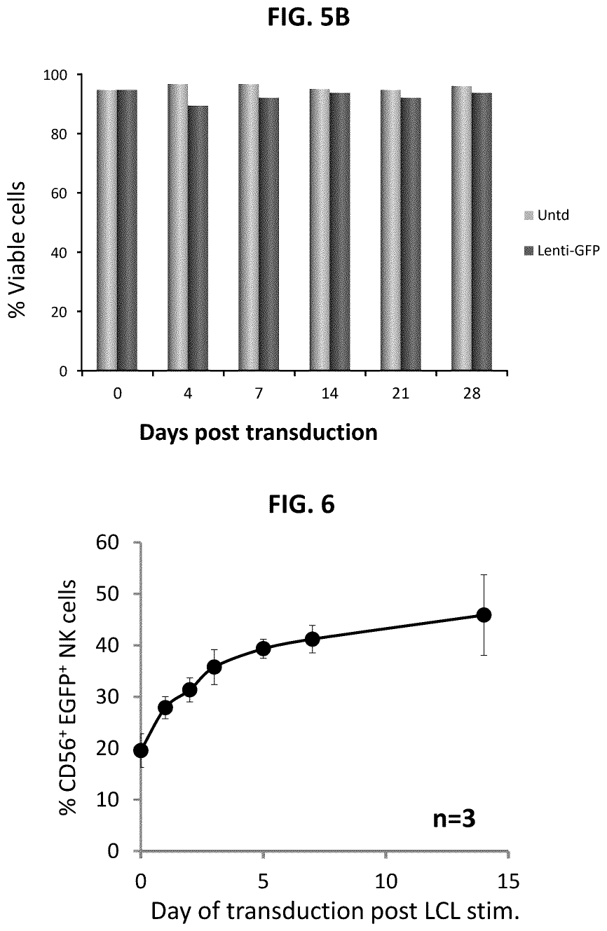

The impact of LCL stimulation on NK cell transduction was also evaluated. NK cells were stimulated with irradiated LCLs in medium containing IL-2 (500 IU/ml) for 1-14 days before transduction. At two days post-transduction, viral particles were removed and the cells were maintained in medium containing 500 IU/ml IL-2. Transduction efficiency increased with the number of days of LCL stimulation prior to transduction (FIG. 6).

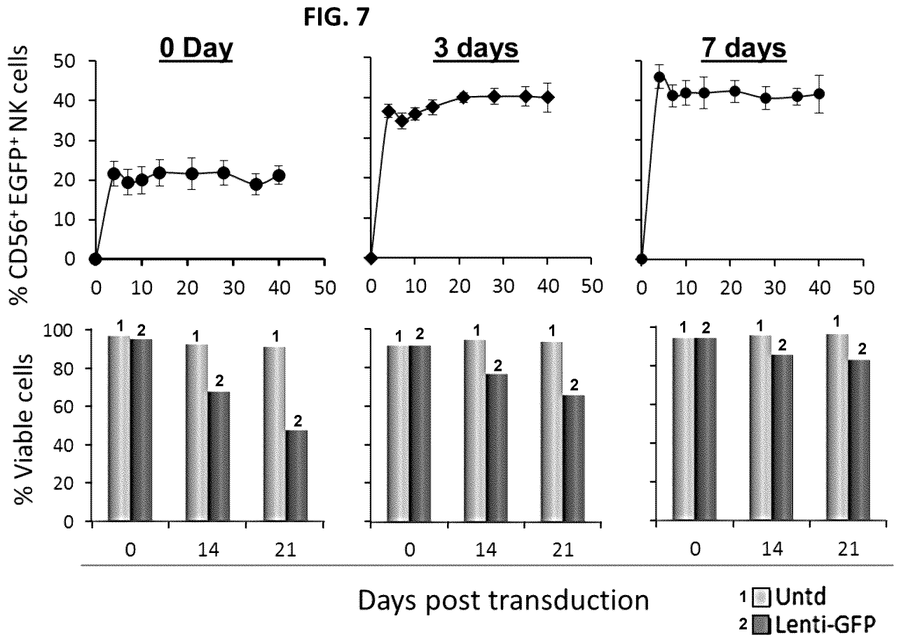

The persistence of transgene expression and cell viability post-transduction following LCL stimulation was tested. NK cells were cultured in medium containing 500 IU/ml IL-2 with irradiated LCLs (10:1 ratio with NK cells) and were transduced on day 0, 3, 7, or 14. At two days post-transduction, viral particles were removed and the cells were maintained in medium containing 500 IU/ml IL-2. eGFP expression remained stable up to 40 days post-transduction under each condition (FIG. 7, top). Cell viability up to 21 days post-transduction improved with priming for 3 days and was highest with priming for 7 days prior to transduction (FIG. 7, bottom).

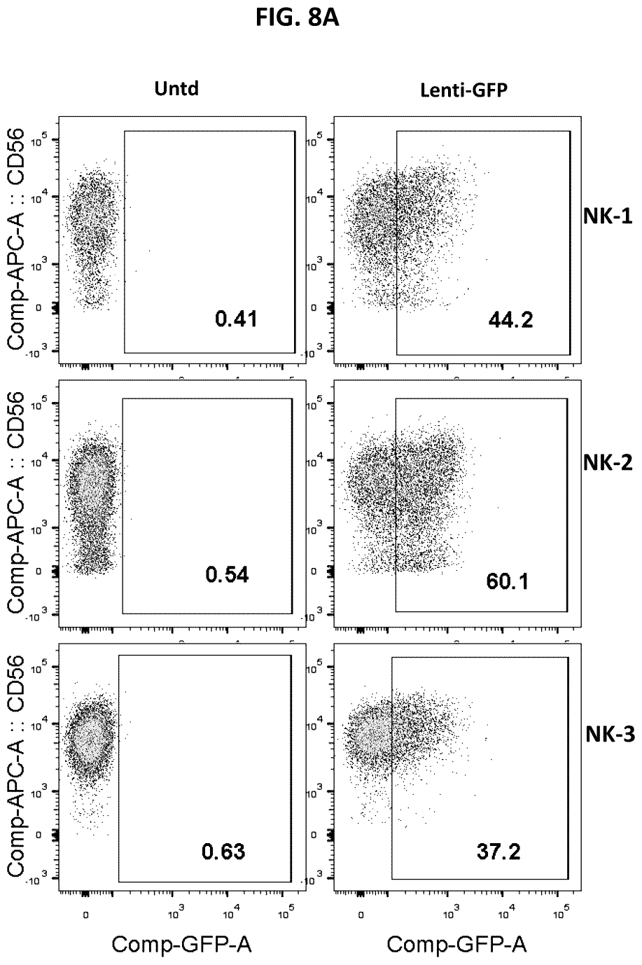

Primary human peripheral NK cells from three subjects were cultured on irradiated LCLs with 500 IU/ml IL-2 for 14 days prior to transduction at MOI 10. Two days post-transduction, viral particles were removed and the cells were maintained in medium containing 500 IU/ml IL-2. eGFP expression was analyzed by FACS at 7 days post-transduction (FIG. 8A) and at additional time points post-transduction (FIG. 8B).

### Example 3

### Effect of IL-2 and IL-15 Priming on Lentiviral Transduction of NK Cells

This example describes the effect of priming with IL-2 and IL-15 on transduction of NK cells using a lentiviral vector and characterization of the transduced NK cells.

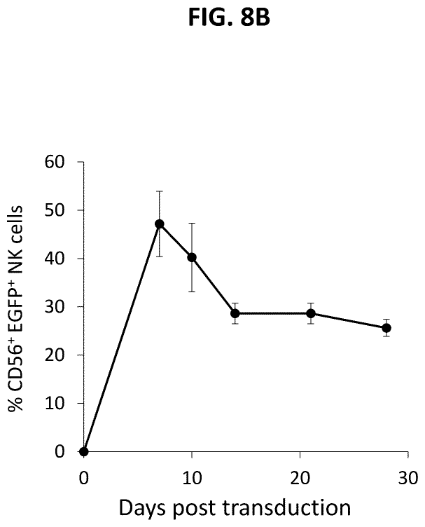

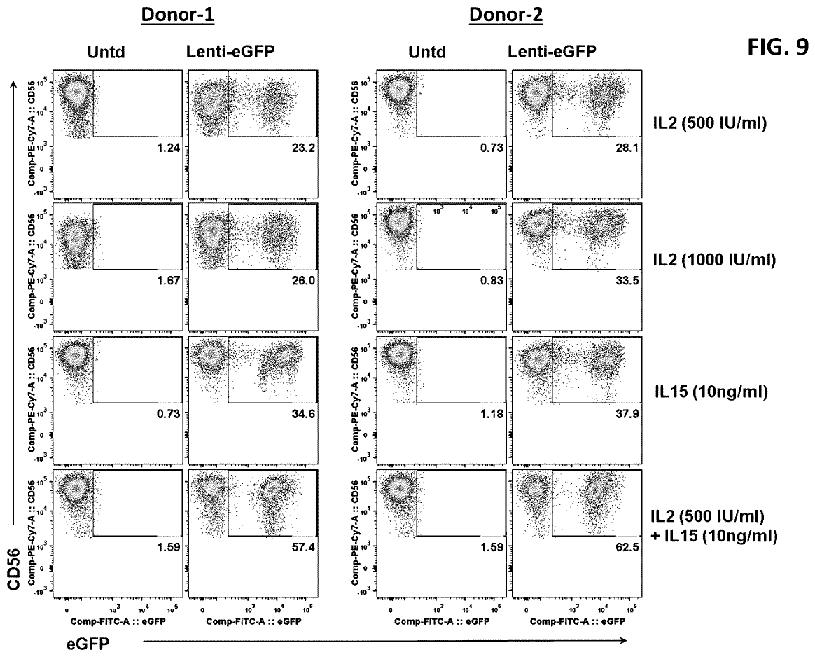

The effect of IL-15 priming, alone or in combination with IL-2, on NK cell transduction was evaluated. NK cells were cultured for 3 days in medium including IL-2 (500 IU/ml or 1000 IU/ml), IL-15 (10 ng/ml), or IL-2 (500 IU/ml) plus IL-15 (10 ng/ml) prior to transduction at MOI 20. Two days post-transduction, viral particles were removed, irradiated LCLs were added (10:1 ratio with NK cells), and the cells were maintained in medium containing 500 IU/ml IL-2. The NK cells primed with IL-2 plus IL-15 were more permissive to transduction than those primed with IL-2 or IL-15 alone (FIG. 9). The cells primed with IL-2 plus IL-15 also maintained long-term expression of the transgene at high frequencies for at least 28 days (FIG. 10).

### Example 4

### Effect of Lentiviral Transduction on NK Cell Phenotype

This example describes the phenotype of lentivirus transduced NK cells.

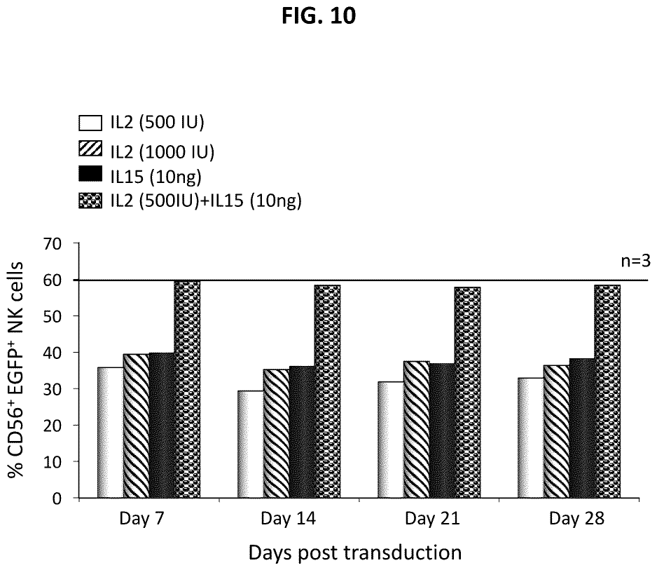

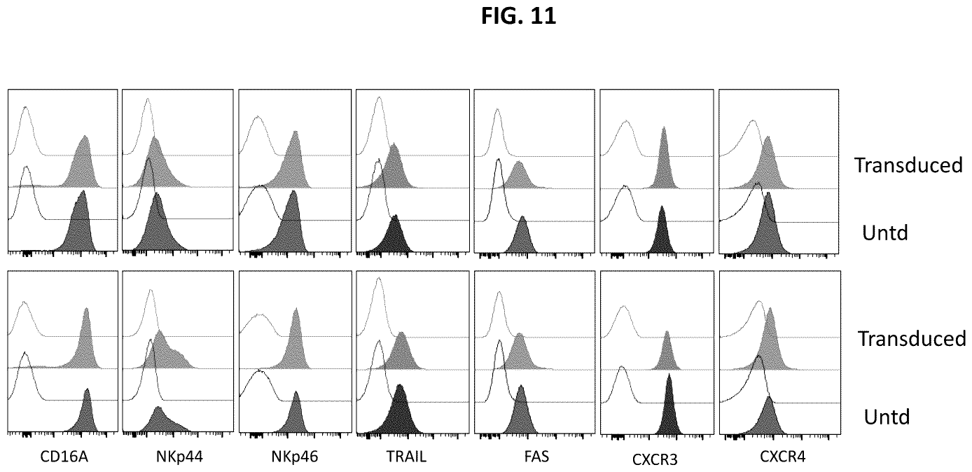

The effect of lentivirus transduction on NK cell phenotype was determined by analyzing expression of receptors clonally expressed on NK cells. NK cells were cultured for 3 days in medium including IL-2 (500 IU/ml) or IL-2 (500 IU/ml) plus IL-15 (10 ng/ml) prior to transduction at MOI 20. Two days post-transduction, viral particles were removed, irradiated LCLs were added (10:1 ratio with NK cells), and the cells were maintained in medium containing 500 IU/ml IL-2. Under both conditions, there was no impact on the NK cell phenotype (FIGS. 11 and 12).

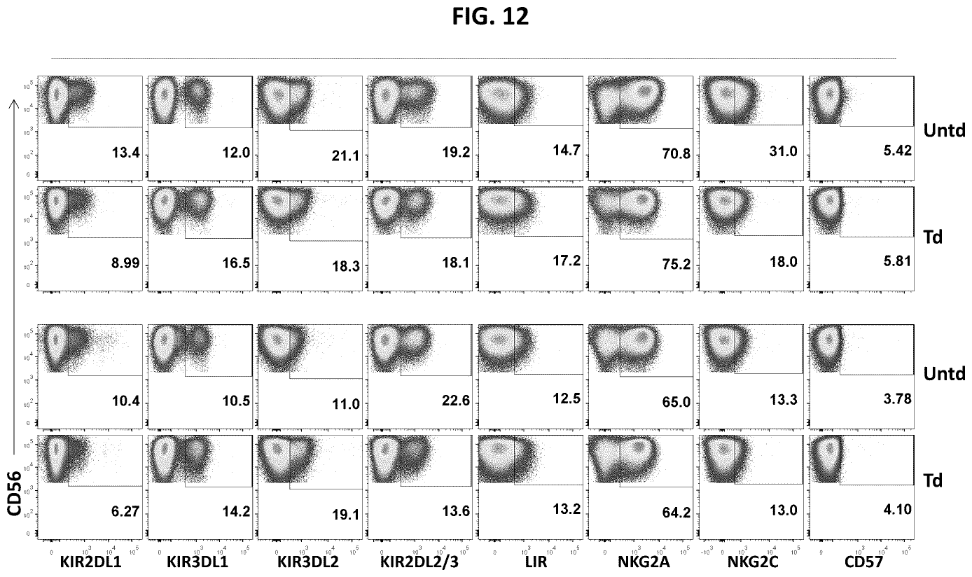

To assess NK cell activity, NK cells from three different healthy donors were primed with irradiated LCL plus 500 IU/ml IL-2, IL-2 alone (500 IU/ml) or IL-2 (500 IU/ml) plus IL-15 (10 ng/ml) for three days prior to transduction. Cells were expanded with irradiated feeder LCLs plus 500 IU/ml IL-2 for 14 days and then their tumor killing capacity was tested against K562 cells (chronic myelogenous leukemia cells) or MOLM14 cells (acute myeloid leukemia cells). The cell killing activity of the transduced NK cells was similar to that of untransduced cells for all priming conditions (FIG. 13).

### Example 5

### Further Evaluation of Culture and Transduction Conditions

This example describes additional experiments evaluating conditions for activation, transduction, and expansion of NK cells.

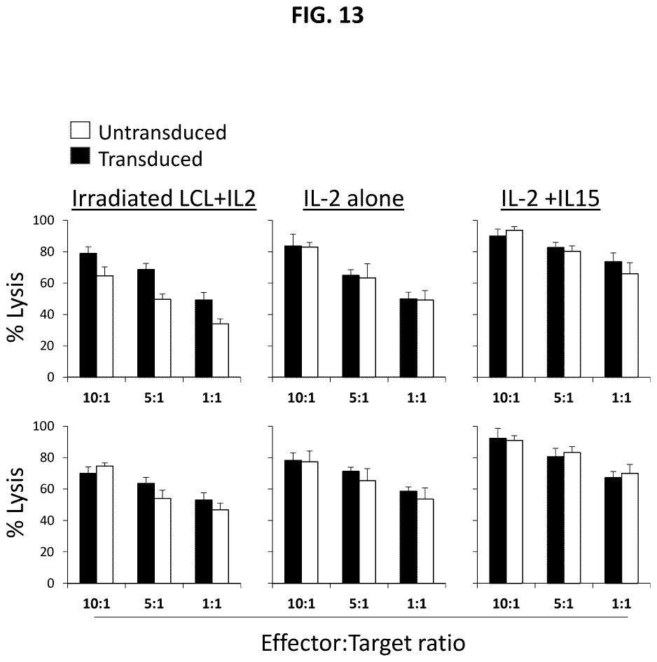

The impact of IL-2 priming on NK cell transduction was evaluated by priming NK cells with 500 IU/ml IL-2 for 1-6 days prior to transduction with a lentiviral vector including a GFP transgene. At two days post-transduction, viral particles were removed and irradiated LCLs were added at a 10:1 ratio (LCL:NK cells) and the cells were maintained in medium containing 500 IU/ml IL-2. Transduction efficiency increased with 1-4 days of priming, then decreased slightly at days 5 and 6 (FIG. 14). Cell viability remained close to 100% with 1-3 days of priming, then dropped off (FIG. 14). Thus, 2-3 days of priming with IL-2 appears to provide the best balance of transduction efficiency and cell viability.

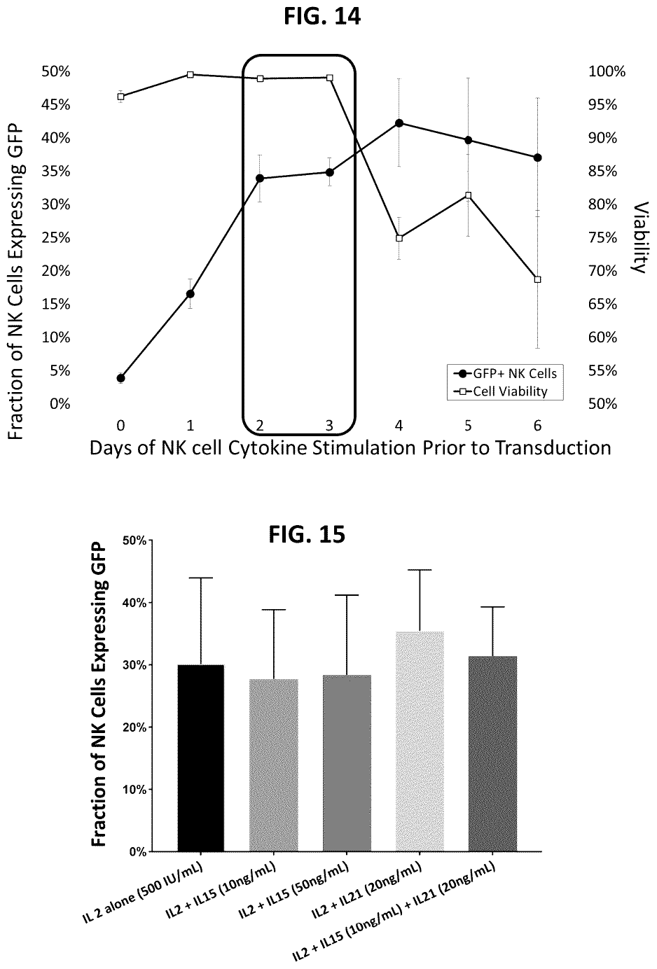

The impact of combinations of cytokines for priming was re-evaluated. NK cells were primed for 3 days with 500 IU/ml IL-2, 500 IU/ml IL-2+10 ng/ml or 50 ng/ml IL-15, 500 IU/ml IL-2+20 ng/ml IL-12, or 500 IU/ml IL-2+10 ng/ml IL-15+20 ng/ml IL-12. At two days post-transduction with a lentiviral vector including a GFP transgene, viral particles were removed and irradiated LCLs were added at a 10:1 ratio (LCL:NK cells) and the cells were maintained in medium containing 500 IU/ml IL-2. No significant differences in transduction efficiency were observed between IL-2 alone and the cytokine combinations (FIG. 15).

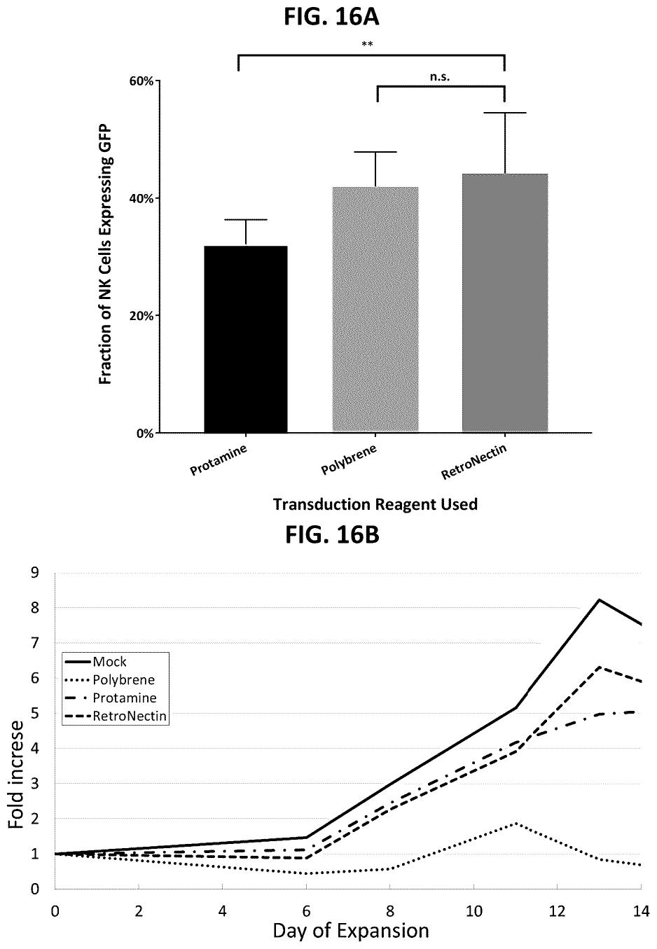

Transduction reagents were also tested for both transduction efficiency and effects on subsequent NK cell expansion. NK cells were primed for 3 days with 500 IU/ml IL-2 and then transduced with a lentiviral vector including a GFP transgene using protamine sulfate, Polybrene®, or Retronectin® reagents. At two days post-transduction, viral particles were removed and irradiated LCLs were added at a 10:1 ratio (LCL:NK cells) and the cells were maintained in medium containing 500 IU/ml IL-2. Including Polybrene® or Retronectin® reagent significantly increased transduction efficiency compared to protamine sulfate (FIG. 16A). Subsequent NK cell viability was significantly decreased in NK cells transduced using Polybrene® reagent compared to protamine sulfate, while viability was not significantly different between NK cells transduced using Polybrene® or Retronectin® reagents (FIG. 16B).

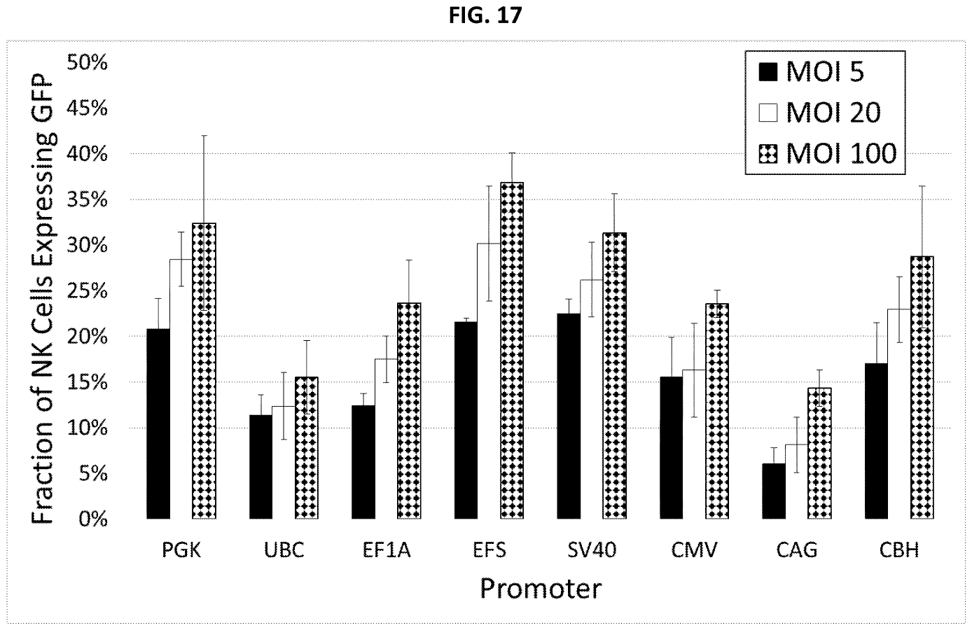

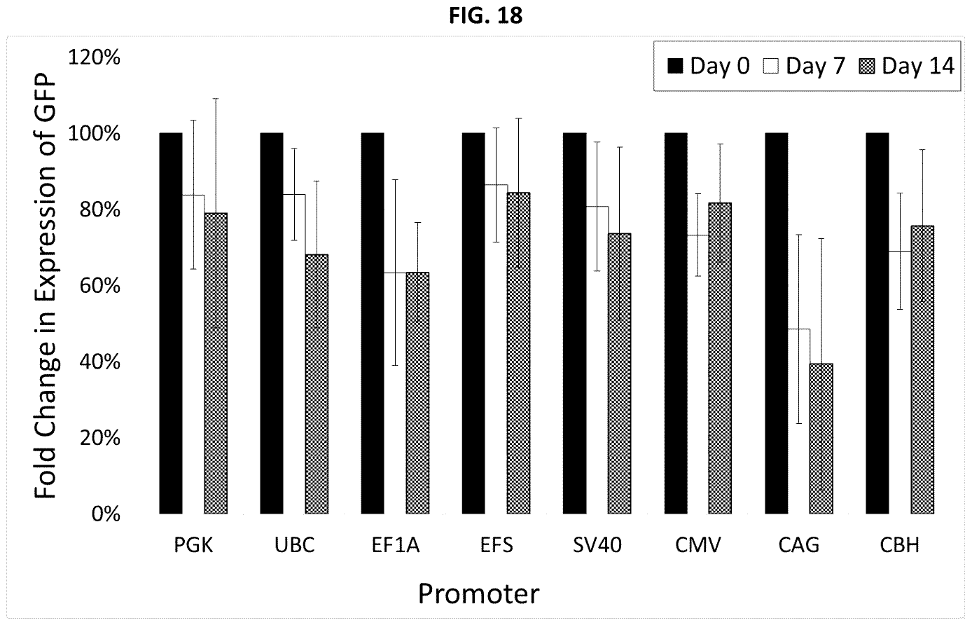

Various promoters were evaluated for NK cell transduction efficiency. Lentiviral vectors including a GFP transgene operably linked to a PGK, UBC, EF1A, EFS, SV40, CMV, CAG, or CBH promoter were constructed. NK cells were primed for 3 days with 500 IU/ml IL-2. At two days post-transduction with the lentiviral vector at MOI of 5, 20, or 100, viral particles were removed and irradiated LCLs were added at a 10:1 ratio (LCL:NK cells) and the cells were maintained in medium containing 500 IU/ml IL-2. All constructs tested resulted in GFP expression in the transduced NK cells (FIG. 17). PGK, EFS, and SV40 primers resulted in the greatest transduction efficiency. In addition, GFP expression was stable over 14 days expansion post-transduction, compared to expression at day 0 (FIG. 18).

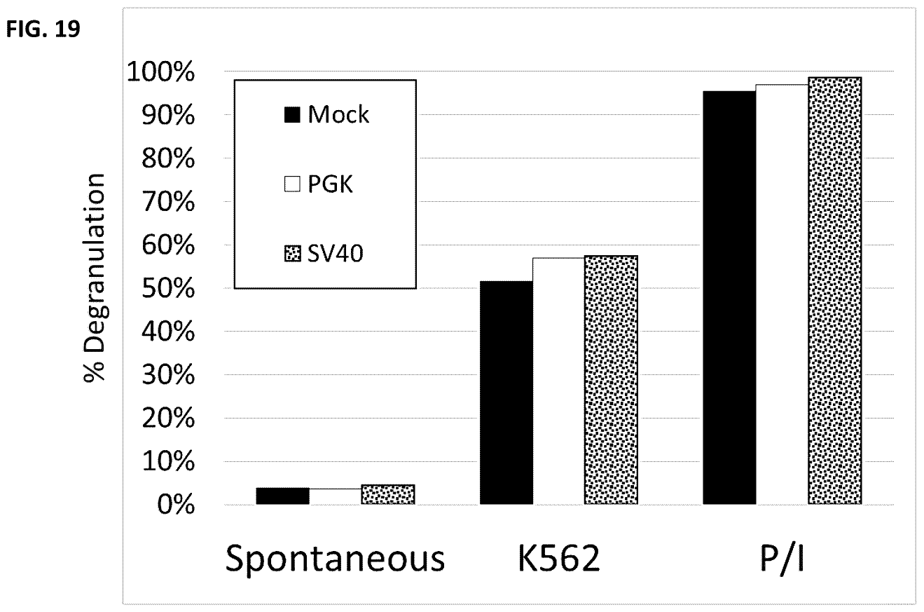

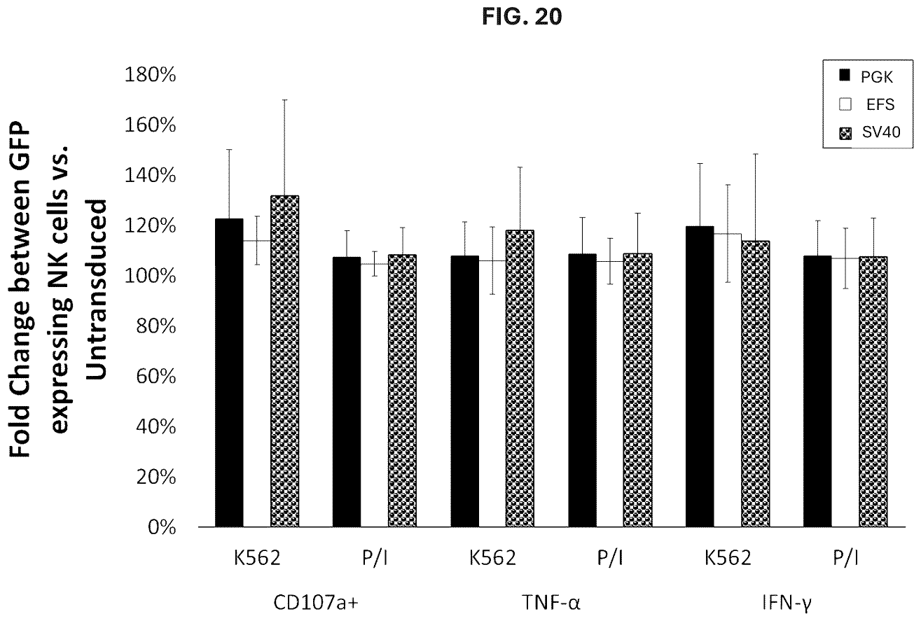

Finally, function of transduced NK cells was evaluated. NK cells were primed for 3 days with 500 IU/ml IL-2. At two days post-transduction with a lentiviral vector including a GFP transgene linked to PGK, EFS, or SV40 promoter, viral particles were removed and irradiated LCLs were added at a 10:1 ratio (LCL:NK cells) and the cells were maintained in medium containing 500 IU/ml IL-2. The transduced cells exhibited equivalent degranulation compared to mock-transduced cells in response to K562 cells, as well as spontaneous and maximal degranulation in response to PMA/ionomycin (FIG. 19). The cells also exhibited equivalent CD107a expression (a marker of degranulation) and IFNγ and TNFα secretion in response to K562 cells or P/I compared to non-transduced cells (FIG. 20).

### Example 6

Transduction of NK Cells with Additional Transgenes

This example describes transduction of NK cells with additional transgenes of interest.

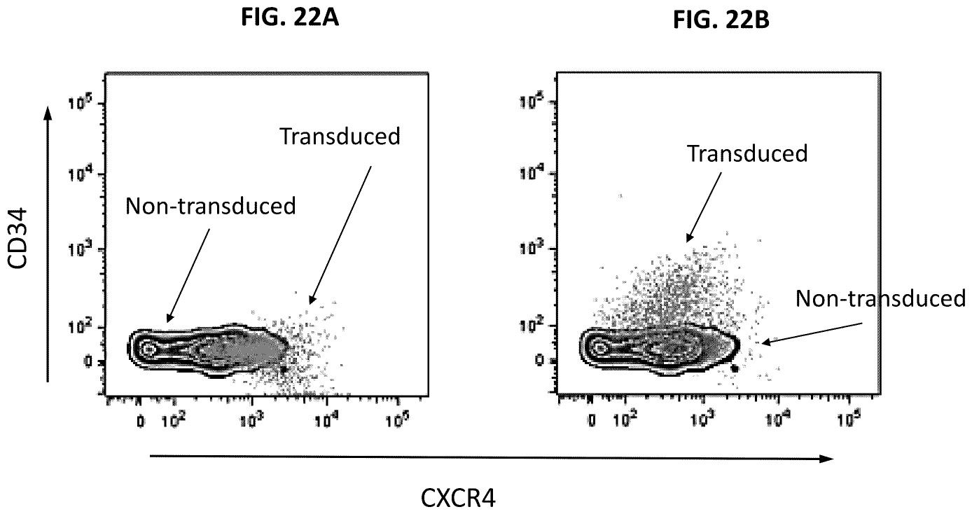

A lentiviral vector including a nucleic acid encoding CXCR4 was constructed (FIG. 21A). NK cells were activated in 500 IU/ml IL-2 for 3 days prior to transduction with MOI 20. Transduced NK cells were expanded for 14 days with irradiated EBV-LCL 2 days following viral transduction. NK cells transduced with this vector showed increased expression of CXCR4 compared to non-transduced cells (FIG. 22A). A lentiviral vector including a nucleic acid encoding CXCR4 linked to a nucleic acid encoding a truncated CD34 molecule including the extracellular and transmembrane domains of CD34, but lacking the intracellular signaling domain was constructed (FIG. 21B). NK cells transduced with this vector (as described above) showed expression of CD34 and increased expression of CXCR4 compared to non-transduced cells (FIG. 22B).

### Example 7

### Evaluation of Transduced NK Cells in a Mouse Model

This example describes methods of evaluating the function and efficacy of modified NK cells expressing a heterologous nucleic acid in a mouse model of hyperproliferative disorder. However, one skilled in the art will recognize that methods that deviate from these specific methods can also be used to evaluate transduced NK cells in an animal model.

A mouse model of myeloma is produced by injecting immunodeficient mice (such as SCID, NOD/SCID, SCID-hu, or SCID-rab mice) with malignant plasma cells or by injecting C57BL/KalwRij mice with allogeneic malignant plasma cells (such as 5T2MM, 5T33, or 5TGM1 cells). In one example, multiple myeloma cell lines are injected in the tail vein of NSG mice and allowed to establish for 7-14 days prior to treating the mice with i.v. infusions of NK cells. Human NK cells are activated (500 IU/ml IL-2 for 2-3 days) and then transduced with a lentiviral vector including a nucleic acid encoding luciferase or CXCR4. Following expansion (14 days on EBV-LCL cells with 500 IU/ml IL-2), the modified NK cells (1-20 million cells) are administered to the mice.

Mice are sacrificed at 7, 14, and 21 days after the NK cell infusion and the % of myeloma cells in the bones (bone marrow flushed from the femurs) is determined to assess the impact of different NK cells (transduced versus non-transduced) on tumor burden. Survival and localization of the modified NK cells may be evaluated by bioluminescent imaging of mice injected with modified NK cells transduced with a luciferase-encoding vector.

### Example 8

Method of Treating a Subject with a Hyperproliferative Disorder

This example describes methods of treating a subject with a hyperproliferative disorder with modified NK cells expressing a heterologous nucleic acid. However, one skilled in the art will appreciate that methods that deviate from these specific methods can also be used to successfully treat or inhibit a hyperproliferative disorder in a subject.

A subject with a hyperproliferative disorder, for example multiple myeloma, undergoes apheresis to collect peripheral blood mononuclear cells. NK cells (e.g., CD56-positive/CD3-negative cells) are isolated from the PBMCs by positive and/or negative selection using immune-magnetic methods. The isolated NK cells are cultured in medium containing 500 IU/ml IL-2 for 2 days. The NK cells are then contacted with lentivirus particles including a viral vector with a heterologous nucleic acid of interest for two days. The heterologous nucleic acid can be CXCR4, high affinity CD16, or both. In some examples, the vector also includes a truncated CD34 nucleic acid. The viral particles are removed and medium containing 500 IU/ml IL-2 and irradiated feeder cells (10:1 ratio feeder cells:NK cells) are added and the NK cells are expanded for 14-28 days. If the vector includes CD34t, prior to addition of IL-2 and feeder cells, CD34-expressing NK cells are enriched using CD34+ immuno-magnetic bead selection. The expanded NK cells can be cryopreserved for later use or can be formulated for administration to the subject (for example, in a pharmaceutically acceptable carrier). A composition comprising 10⁶ to 10¹² of the expanded NK cells is administered to the subject intravenously. Patients with multiple myeloma expressing CD38 may also be treated with an anti-CD38 antibody such as daratumumab. The response of the subject's tumor is monitored for up to 5 years.

### Example 9

### Lentiviral Expression Vectors

Lentiviral expression vectors were constructed using Vectorbuilder design service (Cyagen Biosciences, Inc., Santa Clara, CA). The vectors included different promoters and EGFP or PGK promoter and different human proteins. The vectors have the sequences of SEQ ID NOs: 8-17, as shown in Table 3. The additional vector components and their positions in each sequence are shown in Table 4.

TABLE 3
Lentiviral expression vectors

| Vector Name | Inserted Sequences (Nucleotide positions) | SEQ ID NO: |
|---|---|---|
| pLV[Exp]-CMV > EGFP | Human cytomegalovirus immediate early promoter | 8 |
| | (1959-2547) and EGFP (2578-3297) | |
| pLV[Exp]-EF1A > EGFP | Human eukaryotic translation elongation factor 1α | 9 |
| | (EF1A) promoter (1959-3137) and EGFP (3168-3887) | |
| pLV[Exp]-EFS > EGFP | Short version of EF1A promoter (1959-2190) and | 10 |
| | EGFP (2221-2940) | |
| pLV[Exp]-CAG > EGFP | Cytomegalovirus early enhancer fused with chicken | 11 |
| | β-actin (CAG) promoter (1959-3691) and EGFP | |
| | (3722-4441) | |
| pLV[Exp]-CBH > EGFP | Short version of CAG promoter (1959-2756) and | 12 |
| | EGFP (2787-3506) | |
| pLV[Exp]-SV40 > EGFP | Simian virus 40 early promoter (1959-2302) and EGFP | 13 |
| | (2333-3052) | |
| pLV[Exp]-PGK > EGFP | Mouse phosphoglycerate kinase 1 (PGK) promoter | 14 |
| | (1959-2469) and EGFP (2500-3219) | |
| pLV[Exp]-UBC > EGFP | Human ubiquitin C promoter (1959-3136) and EGFP | 15 |
| | (3167-3886) | |
| pLV[Exp]-PGK > CXCR4 | PGK promoter (1959-2469) and human CXCR4 | 16 |
| | (2500-3558) | |
| pLV[Exp]-PGK > CD34t:T2A:CXCR4 | PGK promoter (1959-2469) and truncated human | 17 |
| | CD34 (2500-3447), T2A peptide (3448-3510), human | |
| | CXCR4 (3511-4569) | |
| pLV[Exp]-EFS > EGFP:P2A:CD4 | Short version of EF1A promoter (1959-2190), EGFP | 18 |
| | (2221-2937), and truncated human CD4 (3004-4278) | |
| pLV[Exp]-EFS > EGFP:P2A:CD19 | Short version of EF1A promoter (1959-2190), EGFP | 19 |
| | (2221-2937), and truncated human CD19 (3004-4005) | |

TABLE 4
Additional vector components

| | SEQ ID NO: | | | | | | | | | |
|---|---|---|---|---|---|---|---|---|---|---|
| | 8 | 9 | 10 | 11 | 12 | 13 | 14 | 15 | 16 | 17 |
| RSV enhancer/ | 1-229 | 1-229 | 1-229 | 1-229 | 1-229 | 1-229 | 1-229 | 1-229 | 1-229 | 1-229 |
| promoter | | | | | | | | | | |
| HIV-1 truncated | 230-410 | 230-410 | 230-410 | 230-410 | 230-410 | 230-410 | 230-410 | 230-410 | 230-410 | 230-410 |
| 5′ LTR | | | | | | | | | | |
| HIV-1 psi | 521-565 | 521-565 | 521-565 | 521-565 | 521-565 | 521-565 | 521-565 | 521-565 | 521-565 | 521-565 |
| packaging signal | | | | | | | | | | |
| HIV-1 Rev | 1075-1308 | 1075-1308 | 1075-1308 | 1075-1308 | 1075-1308 | 1075-1308 | 1075-1308 | 1075-1308 | 1075-1308 | 1075-1308 |
| resp. element | | | | | | | | | | |
| cPPT | 1803-1920 | 1803-1920 | 1803-1920 | 1803-1920 | 1803-1920 | 1803-1920 | 1803-1920 | 1803-1920 | 1803-1920 | 1803-1920 |
| WPRE | 3336-3933 | 3926-4523 | 2979-3576 | 4480-5077 | 3545-4142 | 3091-3688 | 3258-3855 | 3925-4522 | 3597-4194 | 4608-5205 |
| HIV-1 truncated | 4015-4249 | 4605-4839 | 3658-3892 | 5159-5393 | 4224-4458 | 3770-4004 | 3937-4171 | 4604-4838 | 4276-4510 | 5287-5521 |
| 3′ LTR | | | | | | | | | | |
| SV40 early | 4322-4456 | 4912-5046 | 3965-4099 | 5466-5600 | 4531-4665 | 4077-4211 | 4244-4378 | 4911-5045 | 4583-4717 | 5594-5728 |
| polyA signal | | | | | | | | | | |
| Amp^{R} | 5410-6270 | 6000-6860 | 5053-5913 | 6554-7414 | 5619-6479 | 5165-6025 | 5332-6192 | 5999-6859 | 5671-6531 | 6682-7542 |
| pUC Ori | 6441-7029 | 7031-7619 | 6084-6672 | 7585-8173 | 6650-7238 | 6196-6784 | 6363-6951 | 7030-7618 | 6702-7290 | 7713-8301 |

| | SEQ ID NO: | |
|---|---|---|
| | 18 | 19 |
| RSV enhancer/ | 1-229 | 1-229 |
| promoter | | |
| HIV-1 truncated | 230-410 | 230-410 |
| 5′ LTR | | |
| HIV-1 psi | 521-565 | 521-565 |
| packaging signal | | |
| HIV-1 Rev | 1075-1308 | 1075-1308 |
| resp. element | | |
| cPPT | 1803-1920 | 1803-1920 |
| WPRE | 4317-4914 | 4044-4641 |
| HIV-1 truncated | 4996-5230 | 4723-4957 |
| 3′ LTR | | |
| SV40 early | 5303-5437 | 5030-5164 |
| polyA signal | | |
| Amp^{R} | 6391-7251 | 6118-6978 |
| pUC Ori | 7422-8010 | 7149-7737 |

### Example 10

Activation of NK Cells with Feeder Cell Co-Culture

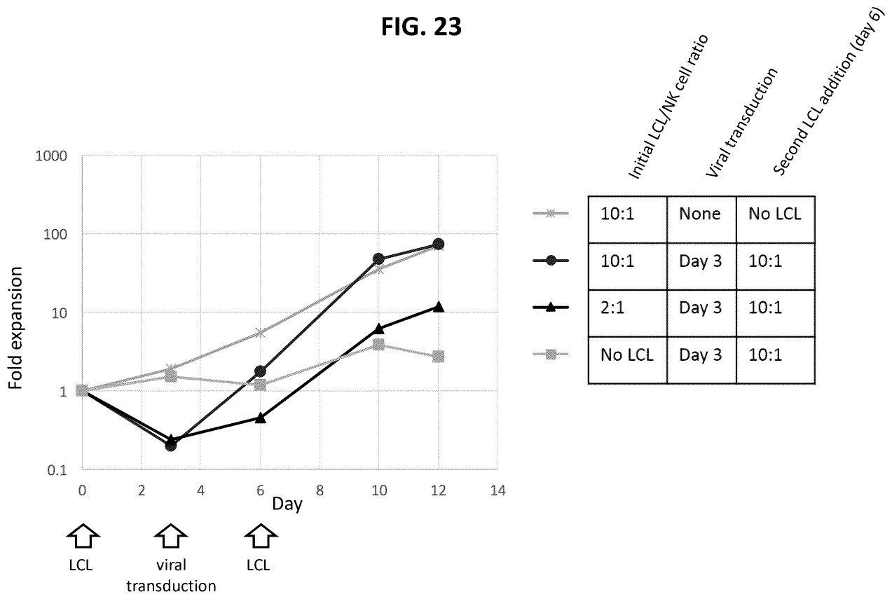

NK cells isolated from human peripheral blood were cultured with or without 100Gy-irradiated SMI-LCL. After three days, cell cultures were centrifuged over Ficoll® media to remove dead cells, counted, and lentivirally transduced with pLV [Exp]-PGK>EGFP:T2A:Luc (SEQ ID NO: 14 with sequences encoding T2A peptide and codon optimized luciferase additionally inserted), using Retronectin® (Takara Clontech) coated plates. After an additional three days, cells were re-counted and re-plated with 10-fold excess of LCL. Cells were maintained throughout in X-VIVO™ 20 media (Lonza) supplemented with 10% heat-inactivated human AB serum, 500 IU/ml IL-2, and 2 mM GlutaMAX™ supplement and counted to determine the fold-expansion. Un-transduced NK cells initially cultured with LCL (10:1 ratio) without subsequent LCL additions were tested for comparison. Cells cultured with a 10-fold excess of LCL prior to transduction had improved expansion compared to cells that were not cultured with LCL prior to transduction and had a similar fold-expansion to un-transduced cells (FIG. 23).

### Example 11

Lentiviral Transduction of Primary NK Cells with IL-2 Culture Prior to Transduction

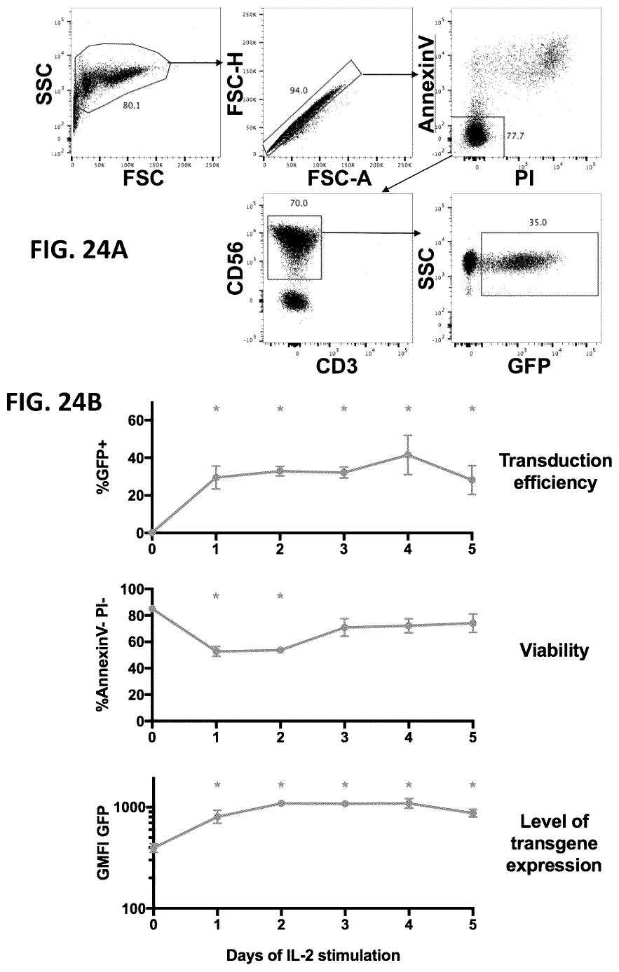

NK cells from PBMC were cultured with 500 U/ml IL-2 for 0 to 5 days before transduction with pLV [Exp]-PGK>EGFP vector at 20:1 MOI in RetroNectin coated plates. Three days after transduction, cells were assayed for GFP expression and viability by flow cytometry. FIG. 24A shows typical gating of GFP+ NK cells. Mean and SEM (of log₁₀ transformed values for geometric mean fluorescence intensity (GMFI) GFP) of experiments from three donors are shown (FIG. 24B). This confirms that activation of NK cells in the presence of IL-2 prior to transduction provides efficient transduction and maintains NK cell viability.

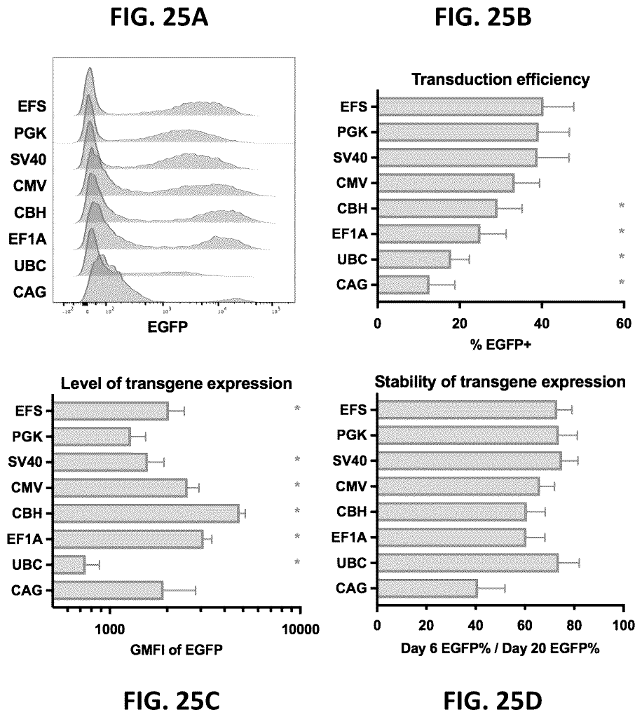

NK cells from PBMC were stimulated with IL-2 for 3 days before transduction with lentiviral vectors containing the indicated promoter sequences and GFP. Three days after transduction, NK cells were examined by flow cytometry for GFP expression (percent and GMFI) (FIGS. 25A-C). Irradiated SMI-LCL feeder cells were added to the transduced NK cells and co-cultured for an additional 14 days (20 days total). Stability of transgene expression in NK cells over time was quantified by dividing the day 6 EGFP % by the day 20 EGFP % and expressed as a percent (FIG. 25D). PGK, EFS, and SV40 primers resulted in the greatest transduction efficiency (FIG. 25B). However, several promoters provided improved levels of transgene expression compared to the PGK promoter, including EFS, SV40, CMV, CBH, and EF1A promoters (FIG. 25C).

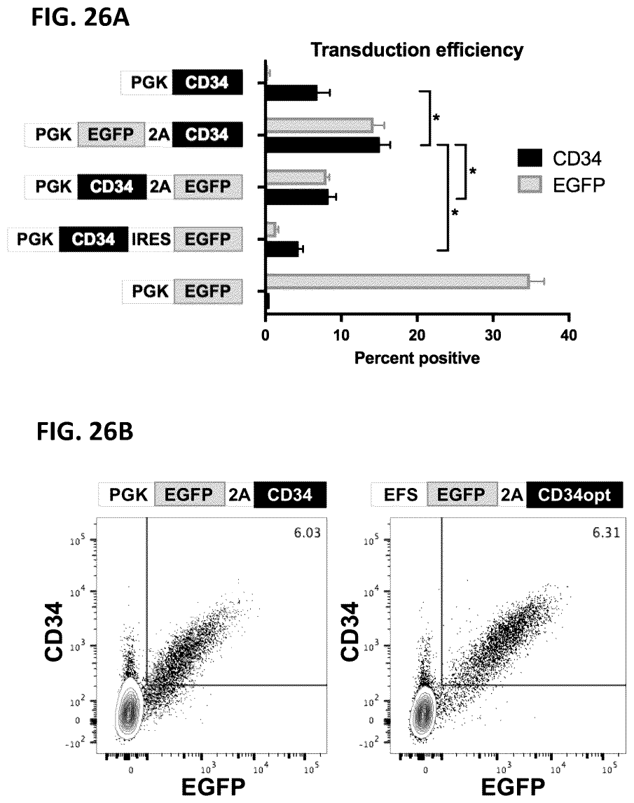

Different combinations of insert sequences within the lentiviral vector were tested for transduction efficiency in human NK cells. NK cells from PBMC were stimulated with IL-2 for 3 days before transduction with lentiviral vectors containing insert sequences encoding combinations of PGK promotor, GFP, P2A-linker, IRES, truncated CD34 protein, and codon-optimized truncated CD34 protein (CD34opt) as shown in FIG. 26A. After 3 additional days, NK cells were measured by flow cytometry for GFP and surface expression of CD34 (FIG. 26A). Transduction of NK cells with a vector encoding CD34opt and the EFS promoter was also tested (FIG. 26B). Placing insert sequences distal to GFP in the vector appeared to enhance their transduction efficiency in human NK cells.

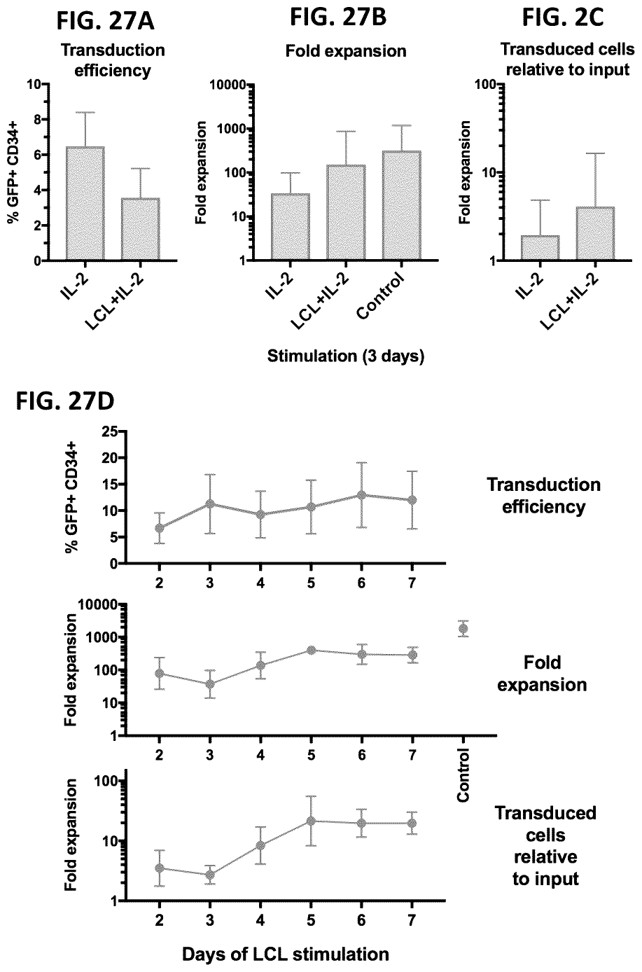

The effect of pre-stimulation of NK cells with IL-2 in the presence of feeder cells prior to transduction was also tested. NK cells from PBMC were stimulated with IL-2 or IL-2+SMI-LCL feeder cells (LCL+IL-2) for 3 days. NK cells were removed from co-culture with anti-CD56 beads before transduction with pLV-EFS-GFP-2A-CD34opt viral particles at 20:1 MOI. After 3 additional days, SMI-LCL were added to both conditions and NK cells cultured until day 21 from the start of experiment. NK cells were examined by flow cytometry for GFP and CD34 transgene expression (FIG. 27A). NK cell numbers were counted frequently throughout culture, and the fold expansion calculated (FIG. 27B). Overall yield of transduced cells relative to input was calculated by multiplying the fold expansion of all cells X the GFP CD34+ fraction for each condition (FIG. 27C). Similar experiments were performed to determine the optimal period (2-7 days) of stimulation with SMI-LCL before transduction (pLV-EFS-GFP-2A-CD34opt at 20:1 MOI). Transduced NK cells received addition of SMI-LCL a second time in each case 3 days after transduction, and cultures were followed until day 21 from the start of the experiment. The percentage of transduced cells, fold expansion of cells, and overall yield of transduced cells relative to input were determined for experiments from 3 donors (FIG. 27D). Pre-stimulation of human NK cells with SMI-LCL feeder cells before lentiviral transduction appeared to enhance the overall yield of transduced cells after NK cell expansion in culture.

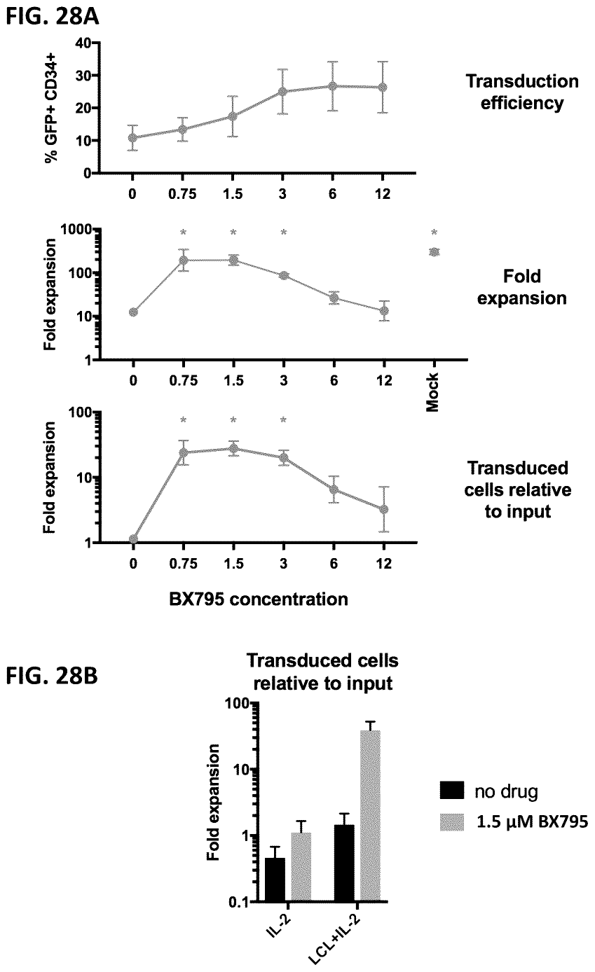

NK cells from PBMC were stimulated with SMI-LCL feeder cells plus IL-2 for 5 days. NK cells were removed from co-culture with anti-CD56 beads before transduction with pLV-EFS-GFP-2A-CD34opt viral particles in the presence of varying doses of BX795. After one day, NK cells were exchanged into medium without BX795. After 2 additional days, SMI-LCL were added again and NK cells cultured until day 21 from the start of experiment, monitoring cell numbers throughout. NK cells were assayed for GFP and CD34 expression by flow cytometry. The overall yield of transduced cells relative to input cells was calculated by multiplying fold expansion of cells X fraction of GFP+CD34+ cells (FIG. 28A). Similar experiments were performed to compare cells stimulated for 5 days with (i) IL-2 or (ii) IL-2+SMI-LCL feeder cells before transduction with or without addition of 1.5 μM BX795 (FIG. 28B). BX795 combined with pre-stimulation with SMI-LCL feeder cells increased subsequent proliferation of lentiviral transduced ex-vivo expanded human NK cells

NK cells were isolated from PBMC via anti-CD3 immunomagnetic bead depletion followed by anti-CD56 immunomagnetic bead positive selection. Cells were stimulated with SMI-LCL plus IL-2 for 4-5 days. Resulting cells were transduced in the presence of 1.5 μM BX795 with lentiviral vectors encoding codon optimized truncated CD34, truncated CD19, or truncated CD4. After 1 day, cells were exchanged into medium without BX795. After 2 additional days, transduced NK cells were selected using immunomagnetic beads recognizing CD34, CD19, or CD4. Positively selected NK cells were cultured with SMI-LCL and proliferation of cells followed until 21 days from the start of the experiments (FIGS. 29A and 29B). Cell numbers were monitored throughout and the overall yield of transduced cells relative to input cells calculated by multiplying the fold expansion of cells X the fraction positive for both GFP and transgene expression.

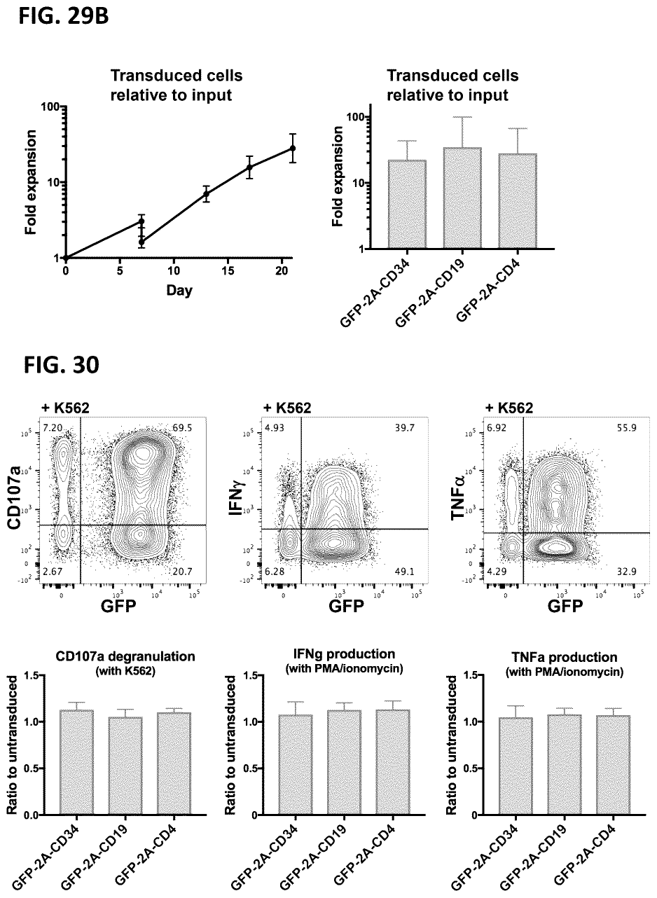

Human NK cells were transduced and expanded as described above for FIGS. 29A and 29B were plated with K562 target cells (1:1 ratio) or treated with PMA plus ionomycin as indicated. Degranulation was assayed after 2 hours with anti-CD107a antibody and cytokine production was measured after 6 hours by intracellular flow cytometry staining for IFN-γ and TNF-α. Lentiviral transduced human NK cells expanded with SMI-LCL feeders showed full degranulation plus IFN-γ and TNF-α production (FIG. 30).

In addition to, or as an alternative to the above, the following embodiments are described:

Embodiment 1 is directed to a method of producing natural killer (NK) cells comprising one or more heterologous nucleic acids, comprising culturing a population of isolated NK cells in the presence of interleukin-2 (IL-2) for at least two days to produce a population of activated NK cells; transducing the population of activated NK cells with a viral vector comprising one or more heterologous nucleic acids to produce a population of transduced NK cells; and culturing the population of transduced NK cells in the presence of IL-2 and irradiated feeder cells to produce an expanded population of transduced NK cells.

Embodiment 2 is directed to the method of embodiment 1, wherein: a) the population of NK cells is cultured in the presence of IL-2 and in the absence of irradiated feeder cells and/or the activated NK cells are transduced with the viral vector in the absence of irradiated feeder cells; or b) the population of NK cells is cultured in the presence of IL-2 and in the presence of irradiated feeder cells and/or the activated NK cells are transduced with the viral vector in the presence of irradiated feeder cells.

Embodiment 3 is directed to the method of embodiment 1 or embodiment 2, wherein culturing the population of isolated NK cells in the presence of one or more cytokines comprises culturing the population of isolated NK cells in cell culture medium containing 500 IU/ml IL-2.

Embodiment 4 is directed to the method of any one of embodiments 1 to 3, wherein the viral vector comprising one or more heterologous nucleic acids is a lentivirus vector.

Embodiment 5 is directed to the method of embodiment 4, wherein the lentivirus vector comprises or consists of a nucleic acid sequence with at least 90% sequence identify to any one of SEQ ID NOs: 8-17.

Embodiment 6 is directed to the method of any one of embodiments 1 to 5, wherein the ratio of the irradiated feeder cells to the transduced NK cells is 10:1.

Embodiment 7 is directed to the method of any one of embodiments 1 to 6, wherein the irradiated feeder cells comprise an Epstein-Barr virus transformed lymphoblastoid cell line or K562 cells.

Embodiment 8 is directed to the method of any one of embodiments 1 to 7, wherein the heterologous nucleic acid encodes: high affinity CD16 (CD16-V158), CXCR4, CCR7, CXCR3, CD34, double negative TGFβ type II receptor, or VLA-4, LFA-1; a chimeric antigen receptor (CAR) that specifically binds to an antigen expressed on tumor cells, wherein the heterologous nucleic acid encodes a CAR that specifically binds to CD19, CD20, CD33, CD138, CS1, GD2, HER2, erbB2-, CEA, EpCAM, NKG2D-L, or TRAIL-R1; a nucleic acid molecule encoding a truncated CD34 protein lacking an intracellular domain; and/or a short hairpin RNA (shRNA), small interfering RNA (siRNA), or an antisense nucleic acid.

Embodiment 9 is directed to the method of embodiment 2b, wherein the population of NK cells is cultured in the presence of IL-2 and in the presence of irradiated feeder cells to produce a population of activated NK cells and the feeder cells are substantially separated from the activated NK cells using CD19 immunodepletion.

Embodiment 10 is directed to 1 method of treating a subject with a tumor, hyperproliferative disease, or viral infection, comprising: obtaining a population of isolated natural killer (NK) cells from the subject or a donor; culturing a population of isolated NK cells in the presence of interleukin-2 (IL-2) for at least two days to produce a population of activated NK cells; transducing the population of activated NK cells with a viral vector comprising one or more heterologous nucleic acids to produce a population of transduced NK cells; culturing the population of transduced NK cells in the presence of IL-2 and irradiated feeder cells to produce an expanded population of transduced NK cells; and administering a composition comprising the expanded population of transduced NK cells to the subject.

Embodiment 11 is directed to the method of embodiment 10, wherein: a) the population of NK cells is cultured in the presence of IL-2 and in the absence of irradiated feeder cells; and/or the activated NK cells are transduced with the viral vector in the absence of irradiated feeder cells; or b) the population of NK cells is cultured in the presence of IL-2 and in the irradiated feeder cells; and/or the activated NK cells are transduced with the viral vector in the presence of irradiated feeder cells.

Embodiment 12 is directed to the method of embodiment 10 or 11, wherein culturing the population of isolated NK cells in the presence of one or more cytokines comprises culturing the population of isolated NK cells in cell culture medium containing 500 IU/ml IL-2.

Embodiment 13 is directed to the method of any one of embodiments 10 to 12, wherein the viral vector comprising one or more heterologous nucleic acids is a lentivirus vector.

Embodiment 14 is directed to the method of embodiment 13, wherein the lentivirus vector comprises or consists of a nucleic acid sequence with at least 90% sequence identify to any one of SEQ ID NOs: 8-17.

Embodiment 15 is directed to the method of embodiment 13, wherein the lentivirus vector comprises a nucleic acid of interest operably linked to a PGK, EFS, or SV40 promoter.

Embodiment 16 is directed to the method of any one of embodiments 10 to 15, wherein the ratio of the irradiated feeder cells to the transduced NK cells is 10:1.

Embodiment 17 is directed to the method of any one of embodiments 10 to 16, wherein the irradiated feeder cells comprise an Epstein-Barr virus transformed lymphoblastoid cell line or K562 cells.

Embodiment 18 is directed to the method of embodiments 10 to 17, wherein the heterologous nucleic acid encodes: high affinity CD16 (CD16-V158), CXCR4, CCR7, CXCR3, CD34, double negative TGFβ type II receptor, or VLA-4, LFA-1; a chimeric antigen receptor (CAR) that specifically binds to an antigen expressed on tumor cells, wherein the heterologous nucleic acid encodes a CAR that specifically binds to CD19, CD20, CD33, CD138, CS1, GD2, HER2, erbB2-, CEA, EpCAM, NKG2D-L, or TRAIL-R1; a nucleic acid molecule encoding a truncated CD34 protein lacking an intracellular domain; and/or a short hairpin RNA (shRNA), small interfering RNA (siRNA), or an antisense nucleic acid.

Embodiment 19 is directed to the method of embodiment 11b, wherein the population of NK cells is cultured in the presence of IL-2 and in the presence of irradiated feeder cells to produce a population of activated NK cells and the feeder cells are substantially separated from the activated NK cells using CD19 immunodepletion.

Embodiment 20 is directed to the method of any one of embodiments 10 to 19, wherein the composition comprising the expanded population of transduced NK cells further comprises a pharmaceutically acceptable carrier.

Embodiment 21 is directed to natural killer cells comprising one or more heterologous nucleic acids produced by the method of any one of embodiments 1 to 9.

Embodiment 22 is directed to a composition comprising natural killer cells comprising one or more heterologous nucleic acids and a pharmaceutically acceptable carrier, wherein the natural killer cells are produced by the method of any one of embodiments 10 to 20.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. Natural killer (NK) cells comprising one or more heterologous nucleic acids, produced by a method comprising:
culturing a population of isolated NK cells in the presence of 500 IU/ml interleukin-2 (IL-2) and in the absence of irradiated feeder cells for at least two days to produce a population of activated NK cells;
transducing the population of activated NK cells with a viral vector comprising one or more heterologous nucleic acids encoding one or more of CXCR4, CXCR3, CD34, double negative TGFβ type II receptor, VLA-4, LFA-1, CD19, or CD4; a CAR that specifically binds to CD19, CD20, CD33, CD138, CS1, GD2, HER2, erbB2, CEA, EpCAM, NKG2D-L, or TRAIL-R1; a truncated CD34 protein lacking an intracellular domain; a short hairpin RNA (shRNA), small interfering RNA (siRNA), or an antisense nucleic acid, to produce a population of transduced NK cells; and
culturing the population of transduced NK cells in the presence of 500 IU/ml IL-2 and irradiated feeder cells to produce an expanded population of transduced NK cells.

2. The NK cells of claim 1, wherein the activated NK cells are transduced with the viral vector in the presence of irradiated feeder cells.

3. The NK cells of claim 1, wherein the population of isolated NK cells is cultured in the presence of IL-2 and no other added cytokines.

4. The NK cells of claim 1, wherein the viral vector comprising one or more heterologous nucleic acids is a lentivirus vector.

5. The NK cells of claim 4, wherein the lentivirus vector comprises or consists of a nucleic acid sequence with at least 90% sequence identity to SEQ ID NO: 16 or SEQ ID NO: 17.

6. The NK cells of claim 1, wherein the ratio of the irradiated feeder cells to the transduced NK cells is about 10:1.

7. The NK cells of claim 1, wherein the irradiated feeder cells comprise an Epstein-Barr virus transformed lymphoblastoid cell line or K562 cells.

8. The NK cells of claim 1, wherein the feeder cells are substantially separated from the activated NK cells using CD19 immunodepletion and/or CD56 immunoselection.

9. Natural killer (NK) cells comprising one or more heterologous nucleic acids, produced by a method comprising:
culturing a population of isolated NK cells in the presence of 500 IU/ml interleukin-2 (IL-2) and irradiated feeder cells for at least two days to produce a population of activated NK cells;
transducing the population of activated NK cells with a viral vector comprising one or more heterologous nucleic acids encoding one or more of CXCR4, CXCR3, CD34, double negative TGFβ type II receptor, VLA-4, LFA-1, CD19, or CD4; a CAR that specifically binds to CD19, CD20, CD33, CD138, CS1, GD2, HER2, erbB2, CEA, EpCAM, NKG2D-L, or TRAIL-R1; a truncated CD34 protein lacking an intracellular domain; a short hairpin RNA (shRNA), small interfering RNA (siRNA), or an antisense nucleic acid, to produce a population of transduced NK cells, wherein the transducing is performed in the presence of one or more inhibitors of toll-like receptor (TLR) or RIG-I-like receptor (RLR) and/or one or more inhibitors of TBK1/IKKε; and
culturing the population of transduced NK cells in the presence of 500 IU/ml IL-2 and irradiated feeder cells to produce an expanded population of transduced NK cells.

10. The NK cells of claim 9, wherein the one or more inhibitors of TLR and/or RLR and/or one or more inhibitors of TBK1/IKKε is one or more of BX795, 2-aminopurine, BAY11-7082, Celastrol, CLI-095, H-89, Norharmane, IRAK1/4 inhibitor, MRT67307, AZ13102909, MPI-0485520, Amlexanox, Compound II, and SR8185.

11. The NK cells of claim 10, wherein the one or more inhibitors of TLR and/or RLR and/or one or more inhibitors of TBK1/IKKε is BX795.

12. The NK cells of claim 9, wherein the activated NK cells are transduced with the viral vector in the presence of irradiated feeder cells.

13. The NK cells of claim 9, wherein the viral vector comprising one or more heterologous nucleic acids is a lentivirus vector.

14. The NK cells of claim 13, wherein the lentivirus vector comprises or consists of a nucleic acid sequence with at least 90% sequence identity to SEQ ID NO: 16 or SEQ ID NO: 17.

15. The NK cells of claim 9, wherein the ratio of the irradiated feeder cells to the transduced NK cells is about 10:1.

16. The NK cells of claim 9, wherein the irradiated feeder cells comprise an Epstein-Barr virus transformed lymphoblastoid cell line or K562 cells.

17. The NK cells of claim 9, wherein the feeder cells are substantially separated from the activated NK cells using CD19 immunodepletion and/or CD56 immunoselection.
